(19) United States
(12) Patent Application Publication
(10) Pub. No.: US 2020/0354348 A1
(43) Pub. Date: Nov. 12, 2020

(54) SEMISYNTHETIC AURONES AND METHODS OF USE THEREOF

(71) Applicant: University of Kentucky Research Foundation, Lexington, KY (US)

(72) Inventors: Jessica S. Blackburn, Lexington, KY (US); Mykhaylo Frasinyuk, Lexington, KY (US); Chunming Liu, Lexington, KY (US); Yanqi Xie, Lexington, KY (US); David S. Watt, Lexington, KY (US)

(21) Appl. No.: 16/963,759

(22) PCT Filed: Jan. 18, 2019
(86) PCT No.: PCT/US19/14299

§ 371 Date: Jul. 21, 2020

Related U.S. Application Data
(60) Provisional application No. 62/620,120, filed on Jan. 22, 2018.

(51) Int. Cl. C07D 405/10; A61P 35/00; C07D 413/10; C07D 417/10; C07D 307/83; C07D 405/06

(52) U.S. Cl. CPC C07D 405/10; A61P 35/00; C07D 405/06; C07D 417/10; C07D 307/83; C07D 413/10

(57) ABSTRACT

Provided herein are semisynthetic aurones, pharmaceutical compositions, and methods of treating cancer. The semisynthetic aurone compound including a structure according to Formula (I) or pharmaceutically acceptable salt thereof, where R¹ is H, an alkyl cyano, an alkyl phenyl substituted with one or more halogens, or any combination thereof, and R² is an aromatic group, a heterocycle, or a combination thereof. The pharmaceutical composition includes the semisynthetic aurone compound and a carrier. The method includes administering to a patient in need of such treatment an effective amount of the semisynthetic aurone compound.

### RELATED APPLICATIONS

This application claims the benefit of U.S. Provisional Application Ser. No. 62/620,120, filed Jan. 22, 2018, the entire disclosure of which is incorporated herein by this reference.

### GOVERNMENT INTEREST

This invention was made with government support under Contract Nos. R00 CA181500; R01 CA172379; DP2 CA228043, and R21 CA205108 awarded by The National Institutes of Health and under Contract Nos. P20 RR020171 and P30 GM110787 awarded by the National Institute of General Medical Sciences. The Government has certain rights in the invention.

### TECHNICAL FIELD

The present disclosure is directed to semisynthetic aurones and methods of use thereof. In particular, the disclosure is directed to semisynthetic aurones having antineoplastic activity and use of such aurones to inhibit cancer cell growth, e.g., leukemias, colon, prostate, breast or lung cancers, in a patient in need thereof.

### BACKGROUND

Aurones comprise a relatively small group of plant-derived flavonoids that arise out of a mixed polyketide-shikimate pathway and that possess a range of biological properties. The antineoplastic activity of several naturally occurring aurones led to studies of natural and semisynthetic aurones as inhibitors of in vitro cancer cell proliferation, typically at low micromolar concentrations. Additional studies related to the antineoplastic activity of these aurones identified various roles at a molecular level: drug efflux modulators of P-glycoprotein (P-gp) or ATP-binding cassette sub-family G member 2 (ABCG2), modifiers of adenosine-receptor interactions, DNA sission-promoters, teleomerase inhibitors, sphingosine-kinase inhibitors, phosphatidylinositol-3-kinases (PI₃-α) inhibitors, cyclin-dependent kinase inhibitors, inducers of cytoprotective NAD(P)H:quinone oxidoreductase-1 (NQO1), and scavengers of reactive-oxygen-species (ROS). However, with such a diverse array of reported effects, care must be exercised to identify the target in biological systems.

Accordingly, there is a continuing need for compounds that can safely and effectively act as antineoplastic agents and treat cancer.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes, in some embodiments, a semisynthetic aurone compound including a structure according to Formula (I):

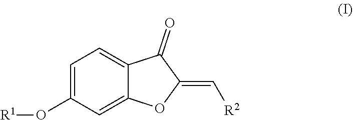

or pharmaceutically acceptable salt thereof, where IV is H, an alkyl cyano, an alkyl phenyl substituted with one or more halogens, or any combination thereof and R² is an aromatic group, a heterocycle, or a combination thereof. In some embodiments, the structure of the compound includes:

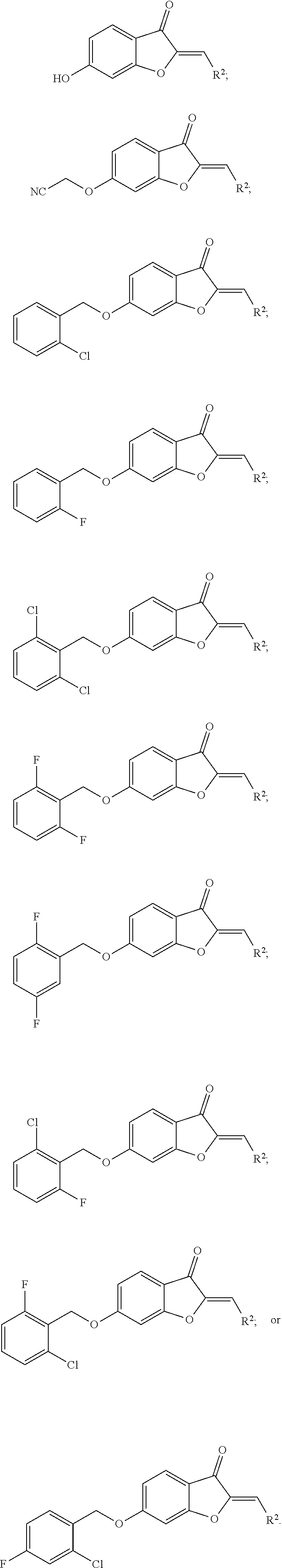

In some embodiments, R² includes an aromatic group. The aromatic group may include at least one substitution, such as, but not limited to, a halogen, an alkyl, an alkoxy group, an amino group, a heterocycle, or any combination thereof. In some embodiments, the compound includes the structure according to Formula (II):

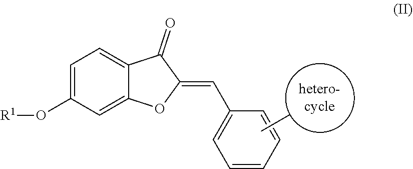

or pharmaceutically acceptable salt thereof. In some embodiments, the heterocycle includes a nitrogen-containing ring. In some embodiments, the nitrogen-containing ring includes a first nitrogen atom and at least one other non-carbon atom, such as, but not limited to, nitrogen, oxygen, sulfur, or any combination thereof. In some embodiments, the heterocycle includes 4 to 10 members. In some embodiment, the heterocycle includes pyridyl, pyridazinyl, pyrrolidyl, morpholinyl, thiomorpholinyl, isoquinolyl, quinolyl, indolyl, or any combination thereof. In some embodiment, the heterocycle includes at least one substitution, such as, but not limited to, O, OH, a halogen, an alkyl, an alkoxy group, an amino group, a carboxyl group, or any combination thereof.

In some embodiments, R² includes a heterocycle. In some embodiments, the compound comprises the structure according to Formula (III):

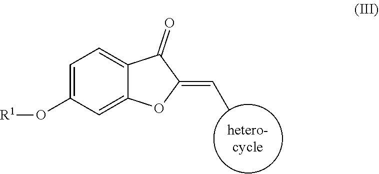

or pharmaceutically acceptable salt thereof. In some embodiments, the heterocycle includes a nitrogen-containing ring. In some embodiments, the nitrogen-containing ring includes a first nitrogen atom and at least one other non-carbon atom, such as, but not limited to, nitrogen, oxygen, sulfur, or any combination thereof. In some embodiments, the heterocycle includes 4 to 10 members. In some embodiment, the heterocycle includes pyridyl, pyridazinyl, pyrrolidyl, morpholinyl, thiomorpholinyl, isoquinolyl, quinolyl, indolyl, or any combination thereof. In some embodiment, the heterocycle includes at least one substitution, such as, but not limited to, O, OH, a halogen, an alkyl, an alkoxy group, an amino group, a carboxyl group, or any combination thereof.

Also provided herein, in some embodiments, is a pharmaceutical composition comprising semisynthetic aurone compound and a carrier.

Further provided herein, in some embodiments, is a method of treating cancer, the method including administering to a patient in need of such treatment an effective amount of the semisynthetic aurone compound. In some embodiments, the cancer includes prostate cancer, colorectal cancer, leukemia, or any combinations thereof.

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-B show a scheme illustrating the formation semisynthetic aurones and images illustrating the structure of various semisynthetic aurones. (A) Scheme illustrating the synthesis of aurones 2-4. Legend: a, heterocyclic-substituted benzaldehydes or heteroarylcarboxaldehydes, 50% aq. KOH, 1:1 EtOH-DMF, b, BrCH₂CN, K₂CO₃, DMF; c, BrCH₂Ar, K₂CO₃, DMF. (B) Images showing the structure of certain semisynthetic aurones according to embodiments of the present disclosure.

FIGS. 2A-B show plots illustrating the effects of certain semisynthetic aurones in inhibiting cancer or tumor growth. (A) Plots of relative inhibition of the proliferation of various cancer cell lines by semisynthetic aurone 4a and its analog 4r. (B) Plots showing that semisynthetic aurone 4a inhibited PC-3 tumor xenografts in nude mice at 10 mg/kg/day, without significant effects on body weights of the treated mice.

FIGS. 3A-B show images and plots relating to the effects of certain semisynthetic aurones. (A) Images illustrating that semisynthetic aurone 4a inhibited tubulin polymerization. PC-3, LS174T and HEK293T cells were treated with 300 nM of 4a. Cell morphologies were changed after 6 h of 4a treatment. (B) Plots illustrating the effects of semisynthetic aurone 4a on cell cycle progression.

FIGS. 4A-E show images and plots relating to the effects of semisynthetic aurone 4a on microtubule structures and tubulin polymerization. (A-C) Images showing that treatment with 4a inhibited microtubule structures in PC-3 cells. Red immunofluorescence: α-tubulin; blue: DAPI. (D) Image showing 4a decreased tubulin polymerization in HEK193T cells. (E) Plot showing 4a (5 μM) inhibits tubulin polymerization in vitro. Colchicine (5 μM) was used as a positive control.

FIGS. 5A-F show images providing information about the structure of semisynthetic aurone 4a and its binding potential. (A) Image showing the chemical structure of 4a. (B) Image illustrating how 4a binds to the colchicine-binding site in the interfaces of αβ-tubulin dimers (cyan for (3, green for a). (C-D) Images providing a close-up view of the interaction environment of 4a (gray sticks) and tubulin (magenta and green ribbon representations). Hydrogen bonding is represented by black dashed lines. (E) Image showing the structure of colchicine. (F) Image showing a superimposition of 4a (gray sticks) and colchicine (yellow lines) in the colchicine-binding site.

FIGS. 6A-G show images and a plot illustrating the effects of semisynthetic aurone 4a and its analog 4r on inhibiting Myc-induced T-ALL in a zebrafish model. (A-C) Images of (A) DMSO, (B) 4a, and (C) 4r treated zebrafish at day 0. (D) Image of DMSO treated zebrafish after 5 days, showing that Myc-induced T-ALL in control fish treated with DMSO as evidenced by development of GFP-labeled thymic lymphoma into T-ALL within 5 days. (E) Image of 4a treated zebrafish after 5 days, showing that the semisynthetic aurone blocked the progress of T-ALL. (F) Image of 4r treated zebrafish after 5 days, showing that the semisynthetic aurone blocked the progress of T-ALL. (G) Plot showing the percent change in leukemia burden in the DMSO and 4r treated zebrafish.

FIGS. 7A-D show plots and images illustrating potential mechanisms of semisynthetic aurones of the present disclosure in inhibiting proliferation of cancer cells using semisynthetic aurone 4a as a model. (A) Plot showing an analysis of NCI60 screening. The cell lines were separated into two groups: sensitive and resistance groups. The protein levels in the two groups were analyzed. The expression of Cyclin A2, encoded by CCNA2 gene, was significantly correlated with 4a response. (B) Image showing 4a induced Cdc2 (CDK1) activation by decreasing phosphorylation at tyrosine 15. (C) Plot showing 4a had synergistic effects with a CDK inhibitor R03306 on the proliferation of LS174T colon and PC3 prostate cancer cells. (D) Image illustrating a mechanism of 4a sensitivity and resistance: 4a inhibits tubulin polymerization and cell cycle progression; cancer cells may develop resistance or feedback rescue mechanisms by activating CDKs. For example, 4a-induced stress may activate Chk1 that in turn inhibits Wee1 and activates cdc25, resulting in a decrease in CDK1 phosphorylation at Y15.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### Definitions

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “semisynthetic” refers to aurones bearing the skeleton found in aurone natural products as well as functional groups and/or additional rings not found in aurone natural products.

All combinations of method or process steps as used herein can be performed in any order, unless otherwise specified or clearly implied to the contrary by the context in which the referenced combination is made.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter relates to semisynthetic aurones, or pharmaceutically acceptable salts or compositions thereof. In some embodiments, the semisynthetic aurones include compounds according to Formula (I):

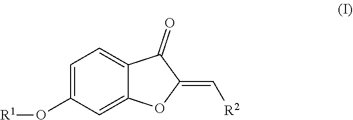

a pharmaceutically acceptable salt thereof, or a pharmaceutical composition thereof. R¹ includes, but is not limited to, H, an alkyl cyano (e.g., a C₁-C₈—CN group), an alkyl phenyl substituted with one or more halogens (e.g., a C₁-C₈-Ph-Xₙ, where Ph is a phenyl group, X represents a halogen such as F, Cl, Br, or I, and n represents an integer of 1-4, preferably 1, 2 or 3), or combinations thereof. R² includes, but is not limited to, an aromatic group (e.g., phenyl, naphthyl, etc.) or a heterocycle such as a heteroaryl group.

Examples of various embodiments including different R¹ groups according to Formula (I) include, but are not limited to:

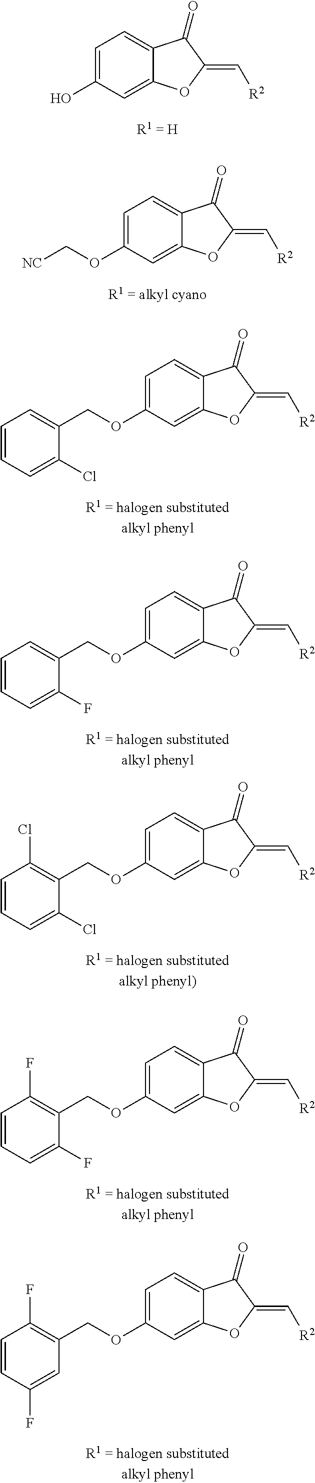

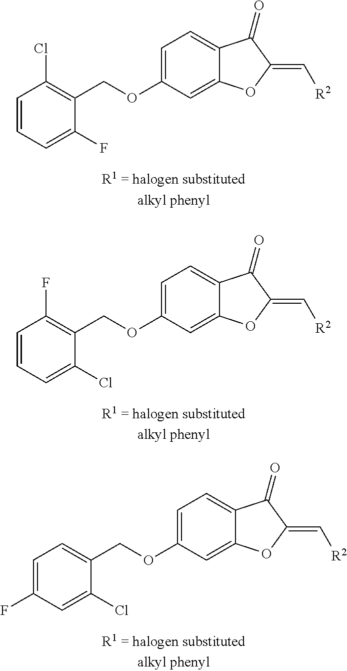

Where R² is an aromatic group, the aromatic group may be unsubstituted or substituted. Suitable substitutions include, but are not limited to, a halogen (e.g., one or more of F, Cl, Br, or I), an alkyl (e.g., C₁-C₈), an alkoxy group (e.g., one or more of OC₁-C₈), an amino group (e.g., alkylamino or dialkylamino group), a heterocycle, or any combination thereof. For example, in some embodiments, the semisynthetic aurones disclosed herein include compounds according to Formula (II):

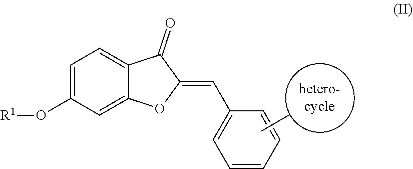

a pharmaceutically acceptable salt thereof, or a pharmaceutical composition thereof. In one embodiment, the heterocycle substituent of the aromatic group is a nitrogen-containing ring. In another embodiment, the nitrogen-containing ring includes a nitrogen atom and at least one other non-carbon atom such as, but not limited to, nitrogen, oxygen, sulfur, or a combination thereof. In a further embodiment, the heterocycle is monocyclic or polycyclic and includes 4-10 members. Suitable monocyclic heterocycles include, but are not limited to, pyridyl, pyridazinyl, pyrrolidyl, morpholinyl, thiomorpholinyl, or a combination thereof. Suitable polycyclic heterocycles include, but are not limited to, fused rings, such as isoquinolyl, quinolyl, indolyl; spirocyclic rings; or a combination thereof. Additionally or alternatively, the heterocycle may be unsubstituted or substituted, such as substituted with one or more of O, OH, a halogen (e.g., one or more of F, Cl, Br, or I), an alkyl (e.g., C₁-C₈), an alkoxy group (e.g., one or more of OC₁-C₈), an amino group (e.g., alkylamino or dialkylamino group), a carboxyl group, or any combination thereof.

Examples of various embodiments including different R² groups according to Formula (II) include, but are not limited to:

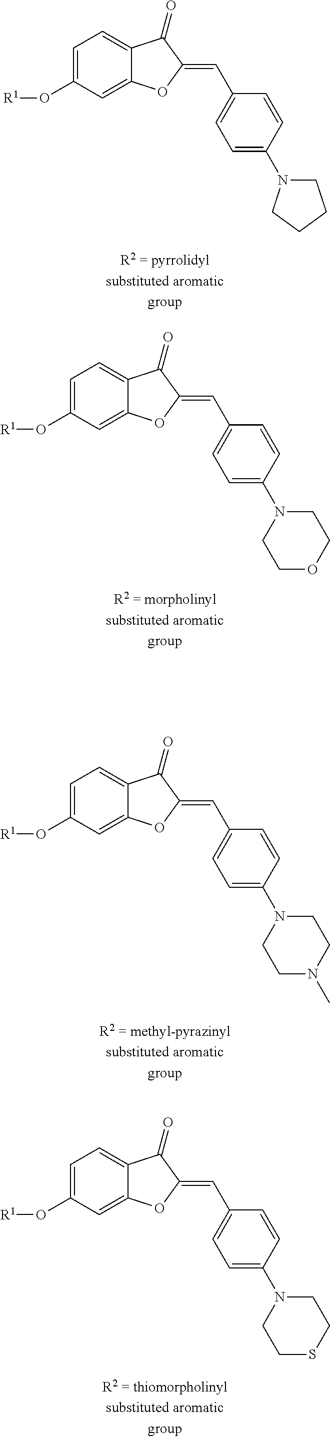

Examples of various embodiments including different non-heterocycle substituted aromatic R² groups include, but are not limited to:

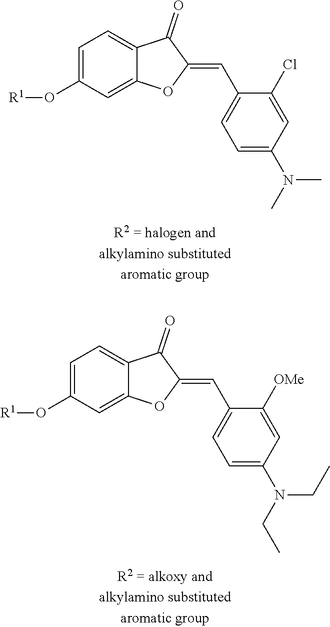

Where R² is a heterocycle, the semisynthetic aurones disclosed herein include, for example, compounds according to Formula (III):

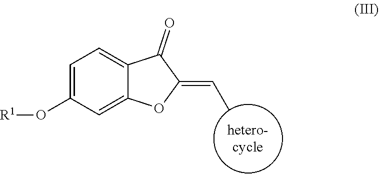

a pharmaceutically acceptable salt thereof, or a pharmaceutical composition thereof. In one embodiment, the heterocycle is a nitrogen-containing ring. In another embodiment, the nitrogen-containing ring includes a nitrogen atom and at least one other non-carbon atom such as, but not limited to, nitrogen, oxygen, sulfur, or a combination thereof. In a further embodiment, the heterocycle is monocyclic or polycyclic and includes 4-10 members. Suitable monocyclic heterocycles include, but are not limited to, pyridyl, pyridazinyl, pyrrolidyl, morpholinyl, thiomorpholinyl, or a combination thereof. Suitable polycyclic heterocycles include, but are not limited to, fused rings, such as isoquinolyl, quinolyl, indolyl; spirocyclic rings; or a combination thereof. Additionally or alternatively, the heterocycle may be unsubstituted or substituted, such as substituted with one or more of O, OH, a halogen (e.g., one or more of F, Cl, Br, or I), an alkyl (e.g., C₁-C₈), an alkoxy group (e.g., one or more of OC₁-C₈), an amino group (e.g., alkylamino or dialkylamino group), a carboxyl group, or any combination thereof.

Examples of various embodiments including different R² groups according to Formula (III) include, but are not limited to:

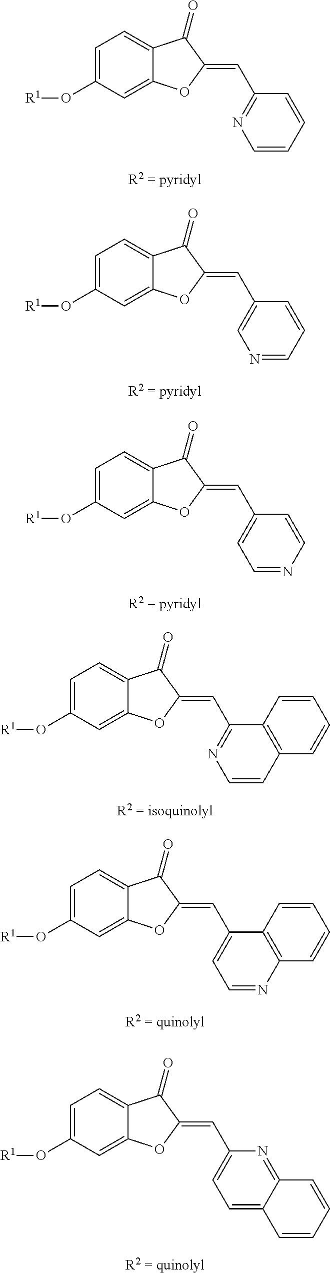

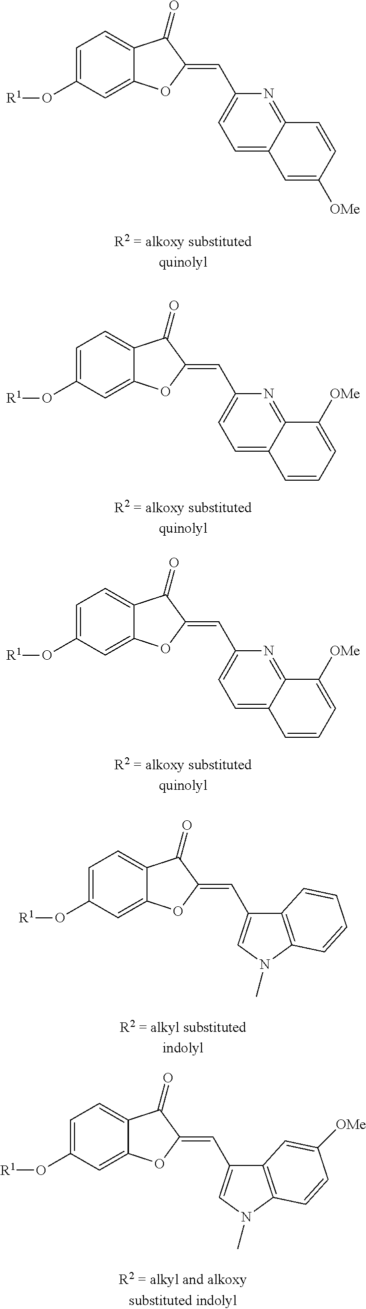

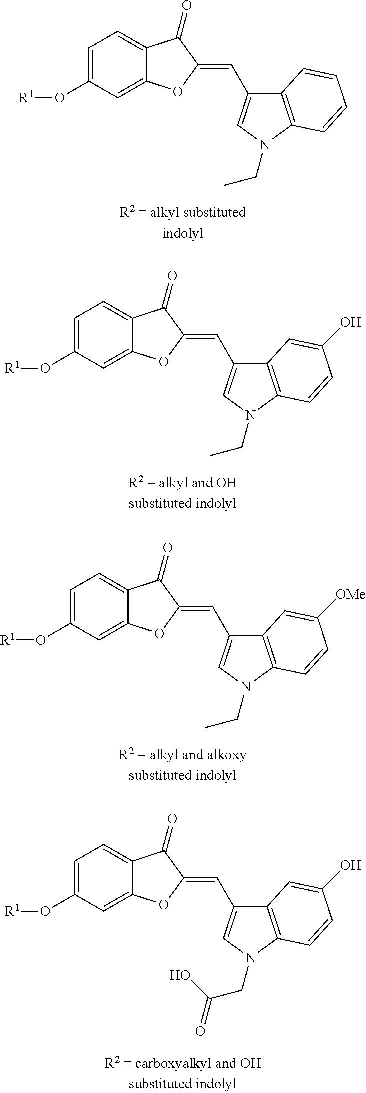

Also provided herein, in some embodiments, is a method of using the semisynthetic aurones disclosed herein. In one embodiment, the method includes administering one of more of the semisynthetic aurone compounds disclosed herein, or a pharmaceutically acceptable salt thereof or a pharmaceutical composition thereof to a subject. In another embodiment, includes administering a pharmaceutically effective amount of the semisynthetic aurone compounds disclosed herein, or a pharmaceutically acceptable salt thereof or a pharmaceutical composition thereof to treat a subject in need thereof. In a further embodiment, the pharmaceutically effective amount of the semisynthetic aurone compounds inhibits cancer cell growth and/or treats cancer. Accordingly, in some embodiments, the subject includes a patient suffering from cancer.

While it may be possible for compounds of the present disclosure to be administered without an additive, in some embodiments, it is preferable to present them as a pharmaceutical composition. According to a further aspect, the present disclosure provides a pharmaceutical composition comprising a compound or mixture of the semisynthetic aurone compounds of Formula (I), or a pharmaceutically acceptable salt, solvate, or hydrate thereof, together with one or more pharmaceutically acceptable additives. Suitable pharmaceutically acceptable additives include, but are not limited to, a pharmaceutically acceptable carrier or excipient and optionally one or more other therapeutic ingredients. The additive(s) must be “acceptable” in the sense of being compatible with the other ingredients of the formulation and not deleterious to the recipient thereof. The term “pharmaceutically acceptable carrier” includes vehicles and diluents.

In some embodiments, the compounds and/or compositions of the present disclosure are useful for treating animals, and in particular, mammals, including humans, as patients. Thus, humans and other animals, and in particular, mammals, suffering from hyperproliferative disorders such as cancer, can be treated by administering to the patient an effective amount of one or more of the semisynthetic aurones according to the present disclosure, or a pharmaceutically acceptable salt thereof, optionally in a pharmaceutically acceptable additive, either alone, or in combination with other known pharmaceutical agents. Treatment according to the present disclosure can also be by administration of the compounds and/or compositions of the present disclosure in conjunction with other conventional cancer therapies, such as radiation treatment or surgery or administration of other anti-cancer agents. In some embodiments, the semisynthetic aurones, or pharmaceutically acceptable salts thereof or pharmaceutically acceptable compositions thereof, target the filamentous, tubulin scaffold. Accordingly, in some embodiments, such compounds are useful for treating a variant of cancers including colorectal cancer and prostate cancer, leukemia, breast cancer, lung cancers.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

Prior to this Example, certain aurones were known but not considered practical as antineoplastic agents because of their limited potency and uncertainty regarding their biological, cellular target(s). In particular, published work led to semisynthetic aurones with varying levels of antineoplastic activity: benzofuran-3(2H)-ones with 2-(coumarin-4-yl)methylene and 2-(furan-2-yl)methylene displayed activity against human leukemia 1(562 cells; benzofuran-3(2H)-ones with a 2-(piperazin-1-yl)methylene possessed IC₅₀ values in the low micromolar range against various solid tumor cell lines; and benzofuran-3(2H)-ones with a 2-(indol-3-yl)methylene inhibited cell proliferation in breast cancer MCF-7 and MDA-MB-231 cell lines. However, the relative potencies among these heteroaryl-substituted aurones and the specific biological target or targets in these cases remains unknown.

With this in mind, structure-activity (SAR) studies involving the modification of the C-2 benzylidene subunit and a C-6 hydroxyl group found in many naturally occurring aurones were conducted. See Scheme 1 below. The “C-2 benzylidene subunit” is also called a “C-2 (phenyl)methylene subunit” and this latter nomenclature will be used not only to describe substituted phenyl systems (e.g., 4-chlorophenyl)methylene but also to describe heterocyclic systems (e.g., (4-pyridyl)methylene).

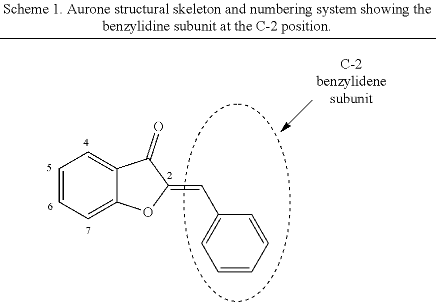 [SMILES: O=C1/C(=C/c2ccccc2)Oc2ccccc21]

In the course of developing the semisynthetic aurones of the present disclosure, several semisynthetic aurones were prepared and tested. The literature indicates two types of microtubule inhibitors that target microtubule dynamics: stabilizing agents, such as paclitaxel, and destabilizing agents, such as the Vinca alkaloids and colchicine. These agents bind tubulin subunits at three well-characterized, binding sites: the taxol-binding site, the Vinca-binding site, and the colchicine-binding site. Agents targeting the taxol- and Vinca-binding sites find broad application in cancer therapeutics; however, agents targeting the colchicine-binding site have not, as yet, found a role in clinical practice, although several candidates are currently in clinical trials. The semisynthetic aurones that are reported here provide a new pharmacophore for the development of a colchicine-targeting microtubule inhibitors for cancer treatment.

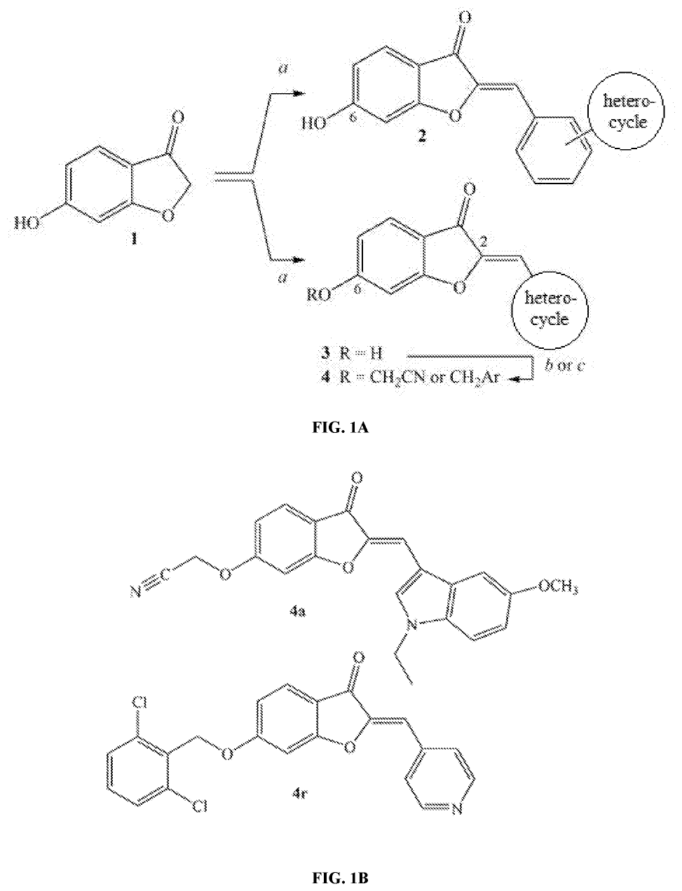

A reiterative process of synthesis and testing guided the identification of C-2 heteroarylmethylene subunits within the aurone pharmacophore that possessed good in vitro inhibitory activity in colon LS174T and prostate cancer PC-3 cell proliferation assays. In particular, heteroaryl-substituted methylene aurones 4 (FIG. 1A) with 2-quinolylmethylene, (8-methoxy-2-quinolyl)methylene, and (5-methoxy-N-ethyl-3-indolyl)methylene groups, respectively, were identified as the most potent analogs at 10 μM concentrations (Table 1).

TABLE 1
Aurones 2 bearing heterocycle-substituted phenylmethylene groups
at C-2 or aurones 3 bearing heteroarylmethylene groups at C-2.

| | | Inhibition of LS174T Cells | | Inhibition of PC-3 Cells | |
|---|---|---|---|---|---|
| Aurone | Ar¹ | 10 μM | 1 μM | 10 μM | 1 μM |
| 2a | 4-(pyrrolidin-1-yl)phenyl | 23 ± 2.9 | 0 | 95 ± 4.7 | 2.1 ± 7.1 |
| 2b | 4-*morpholino-1-yl)phenyl | 0 | | 7.8 ± 7.7 | |
| 2c | 4-(4-methylpyridazin-1-yl)phenyl | 5.6 ± 10 | | 42 ± 14 | |
| 3a | 2-pyridyl | 0 | | 8.8 ± 15 | |
| 3b | 3-pyridyl | 11 ± 3.6 | | 0 | |
| 3c | 4-pyridyl | 19 ± 12 | | 20 ± 5.4 | |
| 3d | 1-isoquinolyl | 79 ± 3.6 | 2.7 ± 6.4 | 88 ± 5.5 | |
| 3e | 2-quinolyl | 96 ± 0.9 | 7.7 ± 9.9 | 99 ± 0.2 | 26 ± 8.6 |
| 3f | 6-methoxy-2-quinolyl | 6.1 ± 13 | | 8.4 ± 33 | |
| 3g | 8-methoxy-2-quinolyl | 91 ± 2.4 | 20 ± 9.1 | 97 ± 0.8 | 40 ± 8.4 |
| 3h | 4-quinolyl | 34 ± 11 | | 63 ± 7.2 | 16 ± 3.5 |
| 3i | 3-indolyl | 44 ± 8.6 | | 20 ± 6.8 | |
| 3j | N-methyl-3-indolyl | 54 ± 15 | | 41 ± 25 | |
| 3k | N-methyl-5-methoxy-3-indolyl | 63 ± 0.7 | 13 ± 2.4 | 35 ± 28 | 17 ± 5.5 |
| 3l | N-ethyl-5-methoxy-3-indolyl | 83 ± 1.2 | 4.5 ± 7.3 | 69 ± 16 | 13 ± 9.5 |

¹Ar in Table 1 refers to the heterocycle-substituted phenyl group on compounds 2 or the heteroaryl group on compounds 3 as shown in FIG. 1.

Modifications of these heteroaryl-substituted methylene aurones 4 at other positions, with one exception, or the replacement of the heteroarylmethylene groups with heterocycle-substituted phenyl groups failed to produce aurones with improved activity relative to the three heteroarylmethylene-substituted aurones 3d, 3e, 3g and 3(1) (Table 1). Among these aurones 3, the indolyl-substituted aurones appeared to be the most promising candidates based on ADME calculations, and additional SAR studies were undertaken to identify still more potent analogs than aurones. Although much of the SAR effort was focused on the indolyl-substituted series, other heteroaryl groups were also investigated.

Alkylation of the C-6 hydroxyl group in aurones 3 with various alkyl bromides secured the heteroaryl-substituted aurones 4 (FIG. 1A). An SAR study involving modifications of the C-6 alkoxy group in aurones 4 and modifications of the C-2 heteroarylmethyelene subunit revealed a synergistic effect favoring either a cyanomethoxy or a 2,6-dichlorobenzyloxy group at C-6 and one of the following C-2 heteroarylmethyelene subunits: N-methyl- or N-ethyl-3-indolylmethylene; 5-methoxy-N-methyl- or 5-methoxy-N-ethyl-3-indolylmethylene; and 4-pyridylmethylene groups. The most relevant portion of these studies is summarized in Table 2.

TABLE 2
SAR study involving modifications of the C-6 alkoxy group in aurones 4 versus
modifications of the C-2 heteroaryl-substituted methylene subunit and using LS174T cell
proliferation assay as a readout.

| | | | C-2 | | | | | | | | |
|---|---|---|---|---|---|---|---|---|---|---|---|
| | | | Heteroaryl | Inhibition of PC-3 Cells | | | | Inhibition of LS174T Cells | | | |
| Aurone | C-6 | C-7 | Group | 10 μM | 1 μM | 300 nM | 100 nM | 10 μM | 1 μM | 300 nM | 100 nM |
| 4a | OCH₂CN | H | N-ethyl-5- | | 95 ± 2.5 | 93 ± 2.8 | 58 ± 11 | 96 ± 0.8 | 97 ± 0.4 | 93 ± 14 | 11 ± 11 |
| | | | methoxy- | | | | | | | | |
| | | | 3-indolyl | | | | | | | | |
| 4b | OCH₂CN | H | N-ethyl-5- | 96 ± 1.6 | 96 ± 2 | 24 ± 11 | | 83 ± 4.8 | 26 ± 15 | | |
| | | | hydroxy- | | | | | | | | |
| | | | 3-indolyl | | | | | | | | |
| 4c | OCH₂CN | H | N-carboxy- | 0 | | | | 83 ± ? | 26 ± 15 | | |
| | | | methyl- | | | | | | | | |
| | | | 5-hydroxy- | | | | | | | | |
| | | | 3-indolyl | | | | | | | | |
| 4d | OCH₂C₆H₄-2-F | H | N-methyl- | 0 | | | | 7.1 ± 17 | | | |
| | | | 3-indolyl | | | | | | | | |
| 4e | OCH₂C₆H₄-2-Cl | H | N-methyl- | 20 ± 6.6 | | | | 0 | | | |
| | | | 3-indolyl | | | | | | | | |
| 4f | OCH₂C₆H₃-2,6-F₂ | H | N-methyl- | 82 ± 4.3 | | | | 0.5 ± 1.7 | | | |
| | | | 3-indolyl | | | | | | | | |
| 4g | OCH₂C₆H₃-2,6-Cl₂ | H | N-methyl- | 96 ± 1.4 | 80 ± 8 | 28 ± 4.4 | | 96 ± 0.2 | 32 ± 2.4 | | |
| | | | 3-indolyl | | | | | | | | |
| 4h | OCH₂C₆H₃-2-F-6-Cl | H | N-methyl- | 31 ± 40 | 0 | | | 38 ± 2 | | | |
| | | | 3-indolyl | | | | | | | | |
| 4i | OCH₂C₆H₃-2,6-Cl₂ | CH₃ | N-methyl- | 7 ± 35 | | | | 28 ± 17 | | | |
| | | | 3-indolyl | | | | | | | | |
| 4j | OCH₂C₆H₃-2-F-4-Cl | H | N-methyl-5- | 8.6 ± 12 | | | | 18 ± 4.2 | | | |
| | | | methoxy- | | | | | | | | |
| | | | 3-indolyl | | | | | | | | |
| 4k | OCH₂C₆H₃-2-F-6-Cl | H | N-methyl-5- | 57 ± 18 | | | | 53 ± 8.9 | | | |
| | | | methoxy- | | | | | | | | |
| | | | 3-indolyl | | | | | | | | |
| 4l | OCH₂C₆H₃-2,6-Cl₂ | H | N-methyl-5- | 96 ± 1.8 | 82 ± 7.7 | 24 ± 7.3 | | 94 ± 18 | 68 ± 0 | 11 ± 3.8 | |
| | | | methoxy- | | | | | | | | |
| | | | 3-indolyl | | | | | | | | |
| 4m | OCH₂C₆H₃-2,6-Cl₂ | CH₃ | N-methyl-5- | 0 | | | | 19 ± 4.6 | | | |
| | | | methoxy- | | | | | | | | |
| | | | 3-indolyl | | | | | | | | |
| 4n | OCH₂C₆H₄-2-F | H | N-ethyl-5- | 52 ± 23 | 28 ± 15 | | | 28 ± 11 | 0 | | |
| | | | methoxy- | | | | | | | | |
| | | | 3-indolyl | | | | | | | | |
| 4o | OCH₂C₆H₃-2,6-Cl₂ | H | N-ethyl-5- | 96 ± 1.1 | 67 ± 5.8 | 3.9 ± 11 | | 94 ± 1.7 | 7.3 ± 18 | 3.3 ± 24 | |
| | | | methoxy- | | | | | | | | |
| | | | 3-indolyl | | | | | | | | |
| 4p | OCH₂C₆H₃-2-F-6-Cl | H | N-ethyl- | 95 ± 4.4 | 91 ± 2.2 | 44 ± 5.6 | | 93 ± 3.2 | 5.1 ± 19 | | |
| | | | 3-indolyl | | | | | | | | |
| 4q | OCH₂C₆H₃-2,6-Cl₂ | H | N-ethyl- | 97 ± 0.6 | 98 ± 1 | 75 ± 7.7 | 17 ± 6.8 | 96 ± 0 | 93 ± 2.5 | 0 | |
| | | | 3-indolyl | | | | | | | | |
| 4r | OCH₂C₆H₃-2,6-Cl₂ | H | 2-pyridyl | | 92 ± 0.4 | 95 ± 1.1 | 76 ± 8.8 | 97 ± 0.3 | 97 ± 0.1 | 95 ± 2.1 | 26 ± 4.3 |

Two candidates displayed significant inhibition (>90%) at 300 nM concentrations: (Z)-2-((2-((1-ethyl-5-methoxy-1H-indol-3-yl)methylene)-3-oxo-2,3-dihydrobenzofuran-6-yl)oxy)acetonitrile (4a) and (Z)-6-((2,6-dichlorobenzyl)oxy)-2-(pyridin-4-ylmethylene)benzofuran-3(2H)-one (4r) (FIG. 1B). C-6 alkoxy groups other than the cyanomethoxy and the 2,6-dichlorobenzyloxy groups possessed less activity than 4a and 4r (Table 2). Modifications of these heteroaryl-substituted aurones 4 with substituents at other positions (e.g., methyl groups at C-7 as in 4i and 4m) also led to compounds with diminished activity.

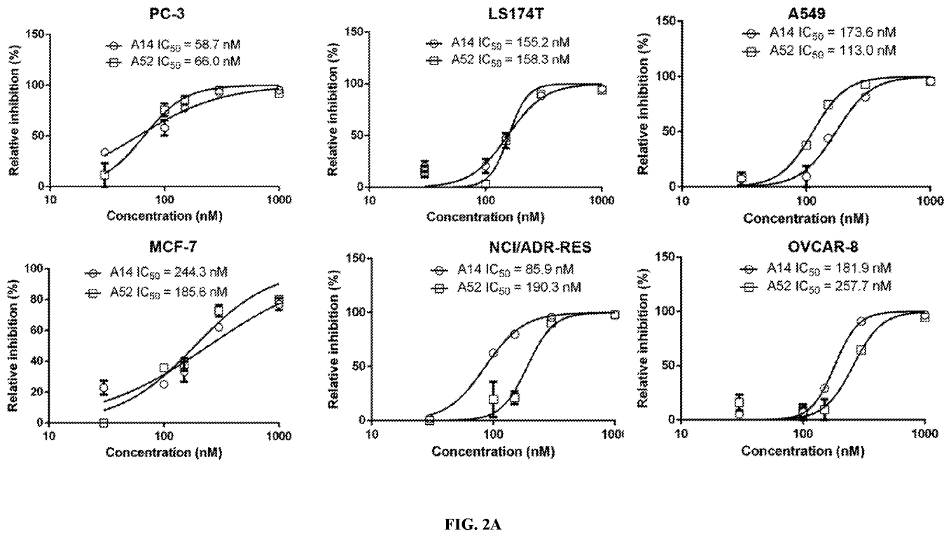

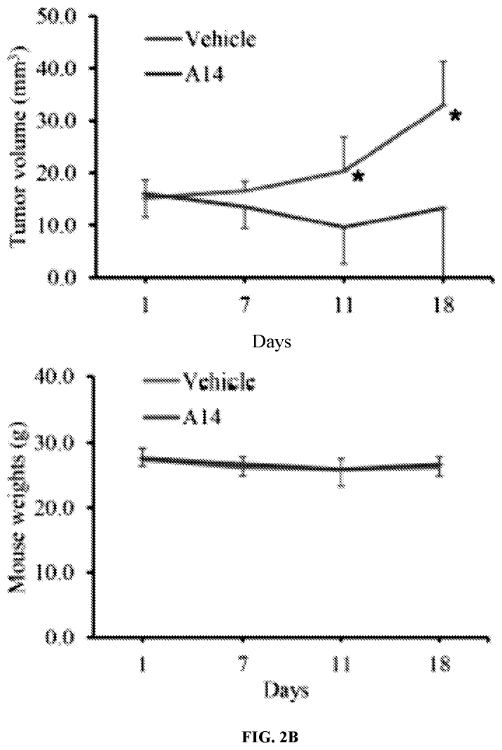

The inhibitory effects of aurones 4a and 4r were determined on various cancer cell lines including prostate cancer PC-3, colorectal cancer LS174T, lung cancer A549, breast cancer MCF-7, and ovarian cancer Ovcar-8 and NCI/ADR-RES (Tables 1-3, FIG. 2A).

TABLE 3
IC₅₀ of 4a in NCI60 cell lines.

| | |
|---|---|
| Panel/Cell Line | GI50 |

Leukemia

| | |
|---|---|
| CCRG-CEM | 289 |
| HL-60(TB) | 236 |
| K-562 | 212 |
| MOLT-4 | 523 |
| RPMI-8226 | 352 |
| SR | 275 |

Non-Small Cell Lung Cancer

| A549(ATCC) | 5.1 | μM |
|---|---|---|
| EKVX | 2.73 | μM |
| HOP-62 | 542 | |
| HOP-92 | NA | |
| NCI-H226 | 57.4 | μM |
| NCI-H23 | 812 | |
| NCI-H322M | 1.43 | μM |
| NCI-H460 | 337 | |
| NCI-H522 | 3.13 | μM |

Colon Cancer

| COLO 205 | 446 | |
|---|---|---|
| HCC-2998 | 3.44 | μM |

| | |
|---|---|
| HCT-116 | 386 |
| HCT-15 | 399 |
| HT29 | 356 |
| KM12 | 546 |

Melanoma

| LOX IMVI | 696 | |
|---|---|---|
| MALME-3M | >100 | μM |
| M14 | 319 | |
| MDA-MB-435 | 174 | |
| SK-MEL-2 | 836 | |
| SK-MEL-28 | 10.2 | μM |
| SK-MEL-5 | 405 | |
| UACC-257 | 67.1 | μM |

| | |
|---|---|
| UACC-62 | 499 |

Ovarian Cancer

| IGROV1 | 774 | |
|---|---|---|
| OVCAR-3 | 377 | |
| OVCAR-4 | 19 | μM |
| OVCAR-5 | 2.52 | μM |

| | |
|---|---|
| OVCAR-8 | 483 |
| NCI/ADR-RES | 406 |
| SK-OV-3 | 669 |

Renal Cancer

| 786-0 | 470 | |
|---|---|---|
| A498 | 10.3 | μM |

| | |
|---|---|
| ACHN | 794 |
| RXF 393 | 182 |
| SN 12C | 763 |

TABLE 4
IC₅₀ of 4a and 4r in leukemia cell lines.

| | | | 95% Confidence |
|---|---|---|---|
| Cell Line | Cell Type | IC50 (nM) | interval (nM) |
| CCRF-CEM | T-ALL | 244 | 197-301 |
| DND41 | T-ALL | 210 | 116-379 |
| Jurkat | T-ALL | 273 | 226-344 |
| HBP-ALL | T-ALL | 94 | 51-173 |
| Loucy | T-ALL | 334 | 285-391 |
| Molt-4 | T-ALL | 241 | 114-402 |
| Molt-16 | T-ALL | 234 | 218-250 |
| RPMI58402 | T-ALL | 301 | 248-364 |
| Nalm-16 | B-ALL | 272 | 248-291 |
| REH | B-ALL | 287 | 252-326 |
| NCI-BL2009 | Normal B-Lymphoblast | 1,253 | 429-3,658 |
| HCC1007-BL | Normal B-Lymphoblast | 1,379 | 372-2,490 |

During a five-day treatment, aurones 4a and 4r showed potent dose-dependent in vitro inhibition of these cancer cell lines with IC₅₀ values in the range of 50-300 nM (FIG. 2A). Most importantly, the in vivo tumor inhibitory effect of aurone 4a was evaluated using prostate cancer PC-3 xenografts in immune-defective nude mice. Compared to vehicle, the administration of aurone 4a at 10 mg/kg/day showed significant, tumor-growth suppression (FIG. 2B). Aurone 4a achieved tumor regression with no apparent gross toxicity as reflected by minimal changes in mice weights (FIG. 2B). In summary, SAR studies identified semisynthetic aurones 4 with specific C-2 heteroarylmethylene and C-6 alkoxy groups that possessed good in vitro activity in cell proliferation studies in the nanomolar range, good reduction in tumor volume in an in vivo prostate PC-3 xenograft study, and minimal gross toxicity based on minimal weight loss during the in vivo studies.

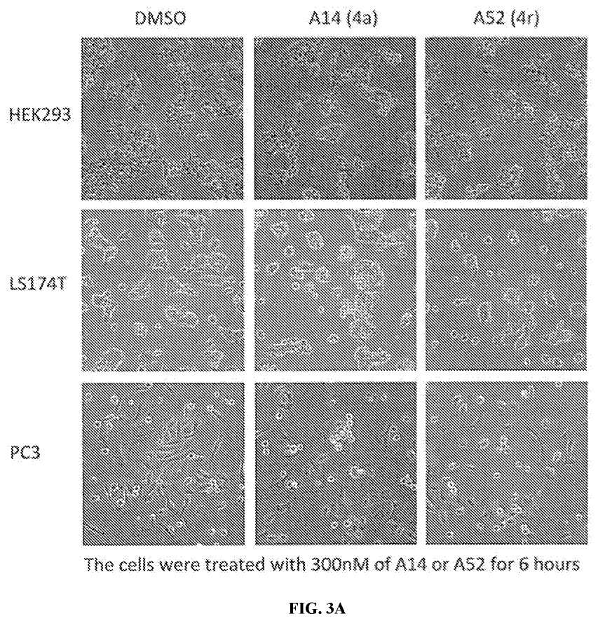

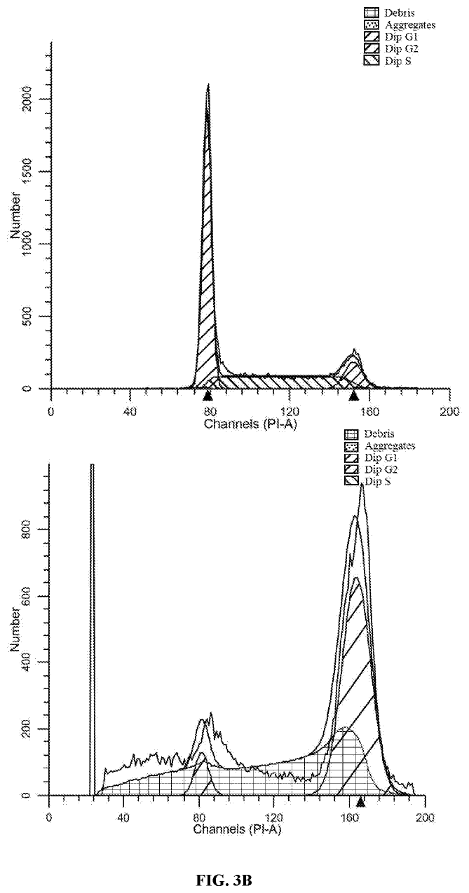

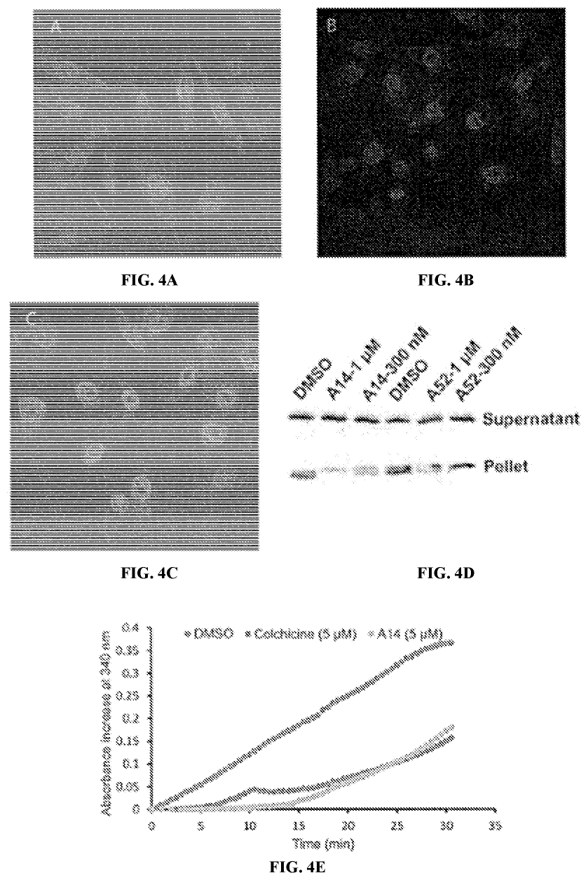

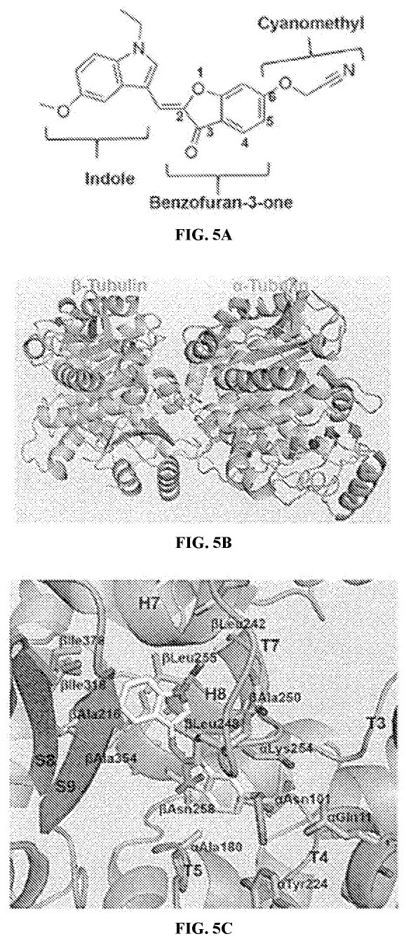

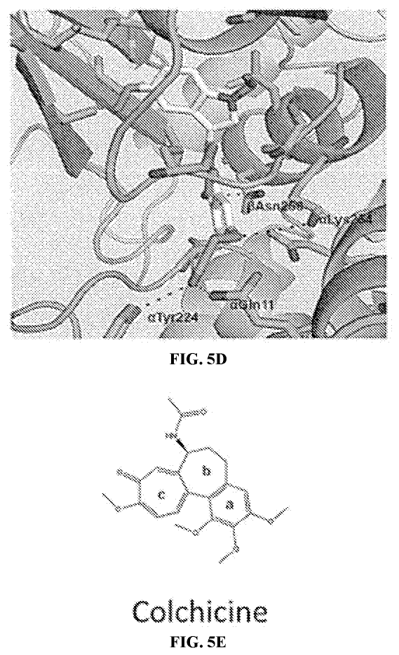

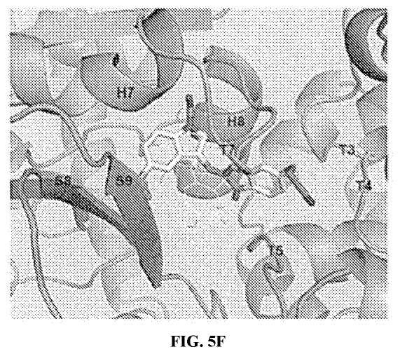

Cell morphology and in vitro tubulin polymerization studies suggested that aurone 4a (FIG. 1B) inhibited cell proliferation by inhibiting tubulin polymerization (FIGS. 3A-4E). NCI-60 screening confirmed that the mechanism of 4a was similar to that of other tubulin inhibitors. Computational docking studies suggested that aurone 4a bound to the colchicine-binding site (CBS) (FIGS. 5A-F). Prior crystallography studies showed that free tubulin dimers transition from a “straight” alignment to a “curved” state in the polymerized microtubules. During these conformation changes, the T7 loop of β-tubulin transitions into the CBS. Colchicine binds to the CBS, prevents the T7 loop flipping towards the CBS, and thus inhibits tubulin polymerization. Importantly, the computational models suggested that aurone 4a possessed a strong interaction with T7 loop (FIG. 5F), which support the hypothesis that aurone 4a possessed a mechanism of action similar to that colchicine. These results also suggested that introducing other chemical moieties to 4a to occupy where the C ring of colchicine bind in the CBS might improve the potency.

Apart from changes in gross morphology and computational studies, the efficacy of aurone 4a was tested in the NCI-60 cell lines and a number of other cell lines where it demonstrated broad-spectrum, anticancer activity (FIG. 2A, Tables 1-4). It is worth noting that the NCI/ADR-RES cell line was resistant to adriamycin and many other cancer chemotherapeutics due to the expression of P-glycoprotein. The results (FIG. 2A, Table 3) indicated that aurone 1a exhibited potent cytotoxicity against cells expressing the multidrug-resistant cell line and was not likely a substrate of P-glycoprotein.

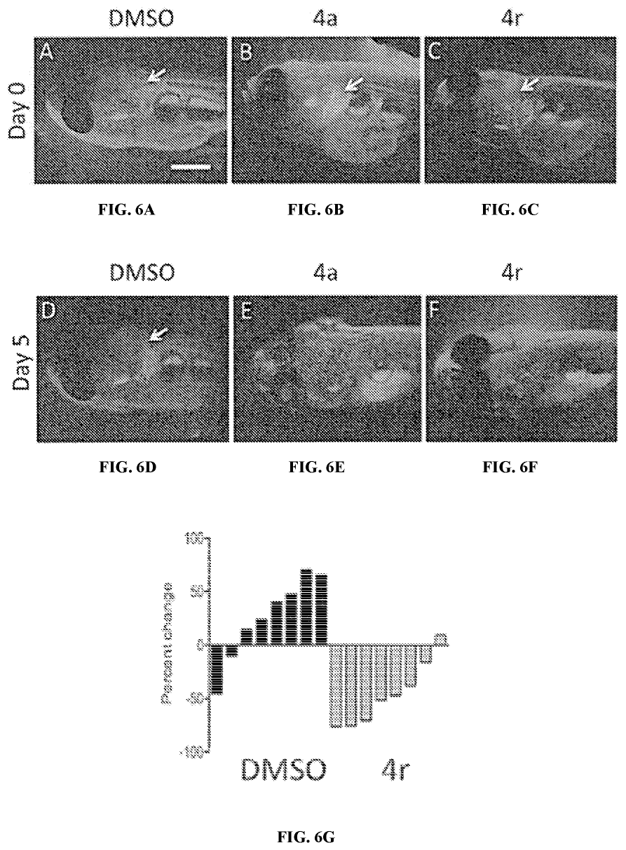

Aurone 4a showed no general toxicity in nude mice at doses that significantly inhibited PC-3 tumor xenografts (FIG. 2B). Aurones 4a and 4r were also tested in zebrafish models where once again no gross toxicity was observed on zebrafish but where significant inhibition of Myc-induced T-ALL was observed in vivo (FIGS. 6A-G). More specifically, DMSO showed a 25.94% increase in leukemia burden, +/−13.87%, while aurone 4a showed a 45.58% decrease in leukemia burden, +/−10.67%. The difference between the groups is significant, at p=0.0011.

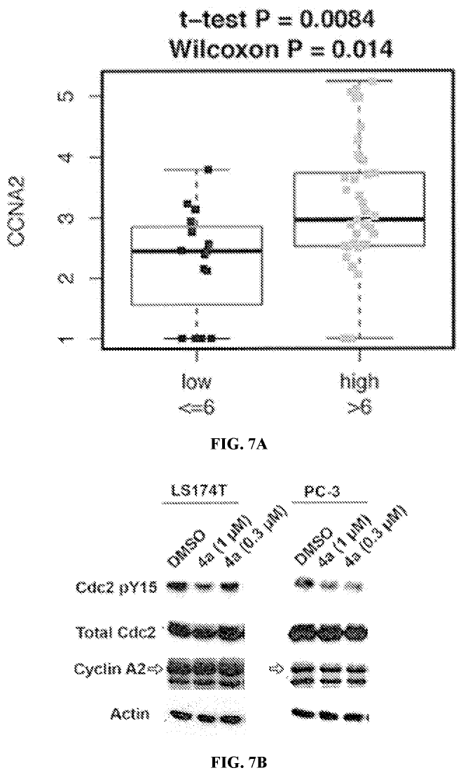

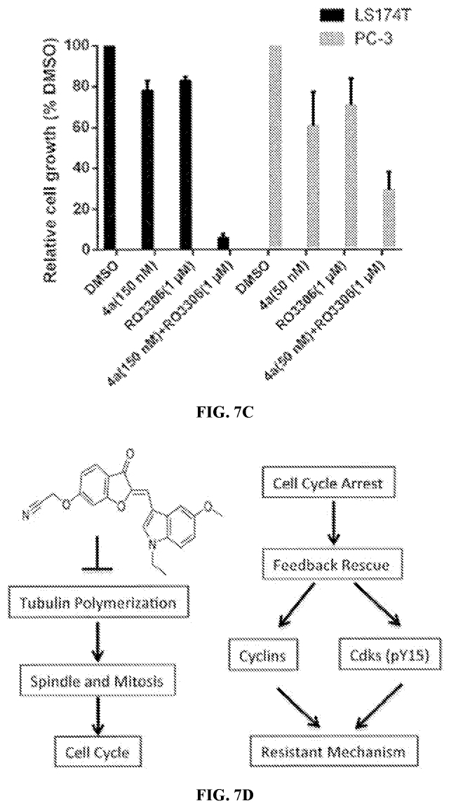

Microtubule inhibitors are highly potent anticancer agents and are widely used in cancer chemotherapy. However, most patients develop drug resistance within a short period of time. In view thereof, the protein expression database of these 60 cell lines was analyzed and it was found that cyclin A2 levels were significantly correlated with 4a sensitivity (FIG. 7A). It was also noted that 4a treatment lead to decrease of Y15 phosphorylation of CDK1. Increasing cyclin A2 or dephosphorylating Y15 of CDK1 leads to activation of cyclin A2/CDK1, which is required for G2/M transition (FIG. 7B). Furthermore, it was found that CDK1 inhibitor, R03306, enhanced 4a effects on inhibiting cancer cell proliferation (FIG. 7C). These results suggested that 4a treatment inhibited mitosis and cells may try to rescue cell cycle progression by enhancing cyclin a2/CDK1 activity (FIG. 7D). These results also suggested that high levels of cyclin A2 or high activity of CDK1 could lead to 4a resistance.

Materials and Methods.

Chemistry

Chemicals were purchased from Sigma-Aldrich (St. Louis, Mo.) or Fisher Scientific (Pittsburgh, Pa.) unless otherwise noted or were synthesized according to literature procedures. Solvents were used from commercial vendors without further purification unless otherwise noted. Nuclear magnetic resonance spectra were determined on a Varian instrument (′H, 400 MHz; ¹³C, 100 Mz). Mass spectra were obtained with an Agilent 1100 (atmospheric pressure chemical ionization) instrument. Melting points were determined in open capillarity tubes with an Buchi B-535 apparatus and are uncorrected. Compounds were chromatographed on preparative layer Merck silica gel F254 unless otherwise indicated.

### (2Z)-6-Hydroxy-2-(4-pyrrolidin-1-ylbenzylidene)-1-benzofuran-3(2H)-one (2a)

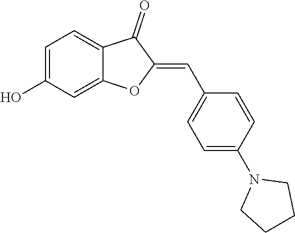 [SMILES: O=C1/C(=C/c2ccc(N3CCCC3)cc2)Oc2cc(O)ccc21]

Yellow crystals (83% yield); mp>220° C.; ¹H NMR (400 MHz, DMSO-d₆) δ 1.83-2.07 (m, 4H), 3.26-3.32 (m, 4H), 6.61 (d, J=8.9 Hz, 2H), 6.66-6.72 (m, 2H), 6.77 (d, J=1.9 Hz, 1H), 7.57 (d, J=8.4 Hz, 1H), 7.77 (d, J=8.9 Hz, 2H), 11 ppm (s, 1H); ¹³C NMR (100 MHz, DMSO-d₆) δ 24.95, 47.24, 98.42, 111.97, 112.58, 112.94, 113.69, 118.62, 125.39, 133.16, 144.73, 148.49, 165.58, 166.87, 180.64 ppm; MS (ACPI) m/z 308.1 (MH⁺, 100).

### (2Z)-6-Hydroxy-2-(4-morpholin-4-ylbenzylidene)-1-benzofuran-3(2H)-one (2b)

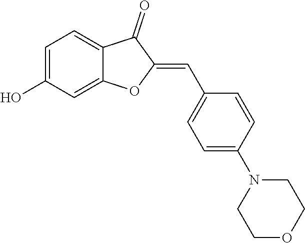 [SMILES: O=C1/C(=C/c2ccc(N3CCOCC3)cc2)Oc2cc(O)ccc21]

Yellow crystals (78% yield); mp>220° C.; ¹H NMR (400 MHz, DMSO-d₆) δ 3.15-3.30 (m, 4H), 3.63-3.80 (m, 4H), 6.70 (dd, J=8.4, 2 Hz, 1H), 6.73 (s, 1H), 6.78 (d, J=1.9 Hz, 1H), 7.02 (d, J=9 Hz, 2H), 7.59 (d, J=8.4 Hz, 1H), 7.82 (d, J=9 Hz, 2H), 11.09 ppm (s, 1H); ¹³C NMR (101 MHz, DMSO-d₆) δ 47.04, 65.89, 98.52, 111.66, 112.78, 113.35, 114.27, 121.93, 125.64, 132.71, 145.65, 151.64, 165.98, 167.29, 181.02 ppm; MS (ACPI) m/z 324.1 (MH⁺, 100).

### (2Z)-6-Hydroxy-2-[4-(4-methylpiperazin-1-yl)benzylidene]-1-benzofuran-3(2H)-one hydrochloride (2c)

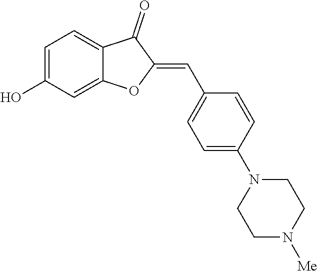 [SMILES: CN1CCN(c2ccc(/C=C3\Oc4cc(O)ccc4C3=O)cc2)CC1]

Yellow crystals (77% yield); mp>220° C.; ¹H NMR (400 MHz, DMSO-d₆) δ 2.83 (s, 3H), 3.04-3.24 (m, 4H), 3.4-3.6 (m, 2H), 3.9-4.2 (m, 2H), 6.72 (dd, J=8.4, 2 Hz, 1H), 6.75 (s, 1H), 6.8 (d, J=2 Hz, 1H), 7.11 (d, J=9.2 Hz, 2H), 7.60 (d, J=8.4 Hz, 1H), 7.86 (d, J=9.2 Hz, 2H), 10.2 (s, 1H), 11.16 ppm (s, 1H); ¹³C NMR (100 MHz, DMSO-d₆) δ 41.9, 44.3, 51.7, 98.5, 111.2, 112.9, 113.2, 115.2, 122.8, 125.7, 132.7, 145.9, 150.1, 166.3, 167.4, 181.1 ppm; MS (ACPI) m/z 337.1 (MH⁺, 100).

### (2Z)-6-Hydroxy-2-[(4-thiomorpholin-4-ylbenzylidene)]-1-benzofuran-3(2H)-one (2d)

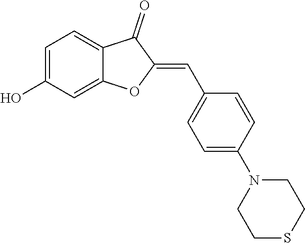 [SMILES: O=C1/C(=C/c2ccc(N3CCSCC3)cc2)Oc2cc(O)ccc21]

Yellow crystals (84% yield); mp>220° C.; ¹H NMR (400 MHz, DMSO-d₆) δ 2.58-2.68 (m, 4H), 3.56-3.86 (m, 4H), 6.7 (dd, J=8.4, 1.9 Hz, 1H), 6.72 (s, 1H), 6.77 (d, J=1.9 Hz, 1H), 7 (d, J=9 Hz, 2H), 7.59 (d, J=8.4 Hz, 1H), 7.81 (d, J=9 Hz, 2H), 11.05 ppm (s, 1H); ¹³C NMR (100 MHz, DMSO-d₆) δ 24.82, 49.69, 98.44, 111.76, 112.72, 113.39, 114.64, 120.9, 125.56, 133.02, 145.47, 150.15, 165.87, 167.16, 180.89 ppm; MS (ACPI) m/z 340.1 (MH⁺, 100).

### (2Z)-2-[2-Chloro-4-(dimethylamino)benzylidene]-6-hydroxy-1-benzofuran-3(2H)-one (2e)

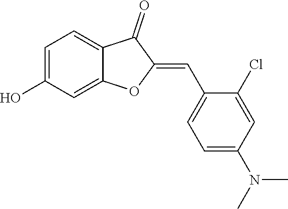 [SMILES: CN(C)c1ccc(/C=C2\Oc3cc(O)ccc3C2=O)c(Cl)c1]

Yellow crystals (83% yield); mp>220° C.; ¹H NMR (400 MHz, DMSO-d₆) δ 3.01 (s, 6H), 6.71 (dd, J=8.4, 2 Hz, 1H), 6.77 (d, J=1.9 Hz, 1H), 6.79-6.84 (m, 2H), 6.95 (s, 1H), 7.6 (d, J=8.4 Hz, 1H), 8.16 (d, J=9.7 Hz, 1H), 11.1 ppm (s, 1H); ¹³C NMR (100 MHz, DMSO-d₆) δ 39.59, 98.52, 106.31, 111.27, 111.86, 112.86, 113.14, 115.87, 125.69, 132.51, 136.25, 145.7, 151.44, 166.02, 167.2, 180.77 ppm; MS (ACPI) m/z 316.2 (MH⁺, 100).

### (2Z)-2-[4-(Diethylamino)-2-methoxybenzylidene]-6-hydroxy-1-benzofuran-3(2H)-one (2f)

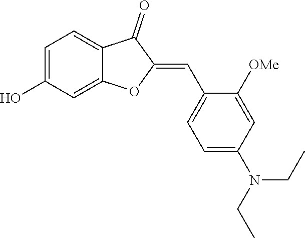 [SMILES: CCN(CC)c1ccc(/C=C2\Oc3cc(O)ccc3C2=O)c(OC)c1]

Yellow crystals (75% yield); mp>220° C.; ¹H NMR (400 MHz, DMSO-d₆) δ 1.15 (t, J=7 Hz, 7H), 3.44 (q, J=7 Hz, 4H), 3.88 (s, 3H), 6.22 (d, J=2.4 Hz, 1H), 6.43 (dd, J=9, 2.4 Hz, 1H), 6.68 (dd, J=8.4, 2 Hz, 1H), 6.75 (d, J=2 Hz, 1H), 7.05 (s, 1H), 7.56 (d, J=8.4 Hz, 1H), 8.03 (d, J=9 Hz, 1H), 10.93 ppm (s, 1H); ¹³C NMR (100 MHz, DMSO-d₆) δ 12.55, 43.96, 55.47, 93.64, 98.34, 104.78, 106.17, 107.43, 112.46, 113.8, 125.26, 132.59, 144.45, 150.55, 160.29, 165.33, 166.56, 180.43 ppm; MS (ACPI) m/z 340.2 (MH⁺, 100).

### (2Z)-6-Hydroxy-2-(pyridin-2-ylmethylene)-1-benzofuran-3(2H)-one (3a)

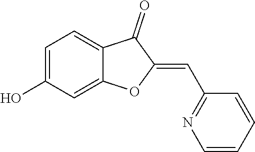 [SMILES: O=C1/C(=C/c2ccccn2)Oc2cc(O)ccc21]

Yellow crystals (93% yield); mp 246-248° C.; ¹H NMR (400 MHz, DMSO-d₆) δ 6.68 (s, 1H), 6.74 (dd, J=8.5, 2 Hz, 1H), 6.81 (d, J=2 Hz, 1H), 7.35-7.46 (m, 1H), 7.66 (d, J=8.5 Hz, 1H), 7.89-7.99 (m, 1H), 8.15 (dd, J=8, 1 Hz, 1H), 8.64-8.74 (m, 1H), 11.35 ppm (s, 1H); ¹³C NMR (101 MHz, DMSO-d₆) δ 98.55, 109.54, 112.25, 113.08, 123.12, 125.63, 125.83, 136.55, 148.58, 149.75, 151.14, 166.75, 168.16, 181.29 ppm; MS (ACPI) m/z 240.0 (MH⁺, 100).

### (2Z)-6-Hydroxy-2-(pyridin-3-ylmethylene)-1-benzofuran-3(2H)-one (3b)

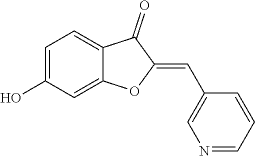 [SMILES: O=C1/C(=C/c2cccnc2)Oc2cc(O)ccc21]

Yellow crystals (83% yield); mp 255-257° C.; ¹H NMR (400 MHz, DMSO-d₆) δ 6.72 (dd, J=8.5, 2 Hz, 1H), 6.81 (d, J=2 Hz, 1H), 6.83 (s, 1H), 7.51 (dd, J=8, 4.8 Hz, 1H), 7.63 (d, J=8.5 Hz, 1H), 8.34 (dt, J=8, 2 Hz, 1H), 8.57 (dd, J=4.8, 2 Hz, 1H), 9.04 (d, J=2 Hz, 1H), 11.3 ppm (s, 1H); ¹³C NMR (125 MHz, DMSO-d₆) δ 98.84, 104.69, 112.14, 113.54, 125.8, 126.15, 130.27, 141.63, 145.08, 146.94, 149.38, 167.29, 168.06, 180.92 ppm; MS (ACPI) m/z 240.0 (MH⁺, 100).

### 2Z)-6-Hydroxy-2-(pyridin-4-ylmethylene)-1-benzofuran-3(2H)-one (3c)

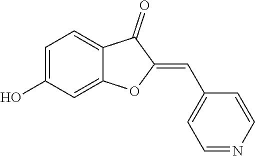 [SMILES: O=C1/C(=C/c2ccncc2)Oc2cc(O)ccc21]

Yellow crystals (91% yield); mp 300-302° C.; ¹H NMR (400 MHz, DMSO-d₆) δ 6.73 (dd, J=8.5, 2 Hz, 1H), 6.76 (s, 1H), 6.82 (d, J=2 Hz, 1H), 7.65 (d, J=8.5 Hz, 1H), 7.79-7.88 (m, 2H), 8.42-8.82 (m, 2H), 11.37 ppm (s, 1H); ¹³C NMR (101 MHz, DMSO-d₆) δ 98.84, 106.96, 112.27, 113.42, 124.37, 126.26, 139.29, 149.8, 150.21, 167.16, 168.27, 181.26 ppm; MS (ACPI) m/z 240.0 (MH⁺, 100).

### (2Z)-6-Hydroxy-2-(isoquinolin-1-ylmethylene)-1-benzofuran-3(2H)-one (3d)

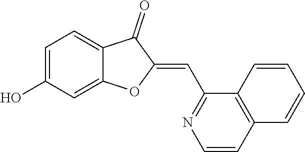 [SMILES: O=C1/C(=C/c2nccc3ccccc23)Oc2cc(O)ccc21]

Yellow crystals (78% yield); mp>220° C.; ¹H NMR (400 MHz, DMSO-d₆) δ 6.71-6.76 (m, 2H), 7.43 (s, 1H), 7.69 (d, J=8.3 Hz, 1H), 7.7-7.77 (m, 1H), 7.79-7.85 (m, 1H), 7.87 (d, J=5.6 Hz, 1H), 8.03 (d, J=8.1 Hz, 1H), 8.35 (d, J=8.9 Hz, 1H), 8.69 (d, J=5.6 Hz, 1H), 11.3 ppm (s, 1H); ¹³C NMR (101 MHz, DMSO-d₆) δ 98.78, 105.12, 112.39, 113.23, 120.99, 125, 126.42, 127.44, 128.25, 130.59, 135.8, 142.65, 149.81, 151.34, 167.15, 169.05, 182.01 ppm; MS (ACPI) m/z 290.2 (MH⁺, 100).

### (2Z)-6-Hydroxy-2-(quinolin-2-ylmethylene)-1-benzofuran-3(2H)-one (3e)

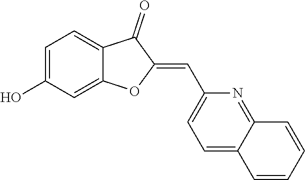 [SMILES: O=C1/C(=C/c2ccc3ccccc3n2)Oc2cc(O)ccc21]

Yellow crystals (72% yield); mp 249-251° C.; ¹H NMR (400 MHz, DMSO-d₆) δ 6.75 (dd, J=8.4, 2 Hz, 1H), 6.78-6.9 (m, 2H), 7.56-7.73 (m, 2H), 7.75-7.88 (m, 1H), 7.93-8.13 (m, 2H), 8.29 (d, J=8.7 Hz, 1H), 8.48 (d, J=8.7 Hz, 1H), 11.39 ppm (s, 1H); ¹³C NMR (126 MHz, DMSO-d₆) δ 98.84, 110, 112.29, 113.41, 122.62, 126.29, 126.88, 127.47, 127.79, 129.08, 130.11, 136.72, 147.82, 149.55, 151.88, 167.13, 168.36, 181.53 ppm; MS (ACPI) m/z 290.0 (MH⁺, 100).

### (2Z)-6-Hydroxy-2-[(6-methoxyquinolin-2-yl)methylene]-1-benzofuran-3(2H)-one (3f)

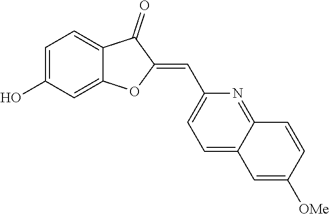 [SMILES: COc1ccc2nc(/C=C3\Oc4cc(O)ccc4C3=O)ccc2c1]

Yellow crystals (70% yield); mp 269-271° C.; ¹H NMR (400 MHz, DMSO-d₆) δ 3.91 (s, 3H), 6.74 (dd, J=8.4, 2 Hz, 1H), 6.77 (s, 1H), 6.83 (d, J=2 Hz, 1H), 7.34-7.48 (m, 2H), 7.65 (d, J=8.4 Hz, 1H), 7.94 (d, J=9 Hz, 1H), 8.24 (d, J=8.7 Hz, 1H), 8.35 ppm (d, J=8.7 Hz, 1H); ¹³C NMR (101 MHz, DMSO-d₆) δ 55.43, 98.57, 105.61, 110.07, 112.12, 113.27, 122.35, 122.71, 125.84, 128.02, 130.45, 135.1, 143.82, 148.85, 149.1, 158.02, 167.07, 168.12, 181.16 ppm; MS (ACPI) m/z 320.0 (MH⁺, 100).

### (2Z)-6-Hydroxy-2-[(8-methoxyquinolin-2-yl)methylene]-1-benzofuran-3(2H)-one (3g)

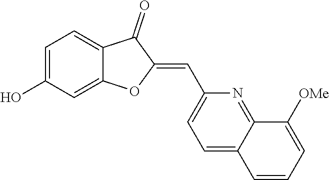 [SMILES: COc1cccc2ccc(/C=C3\Oc4cc(O)ccc4C3=O)nc12]

Yellow crystals (68% yield); mp 250-252° C.; ¹H NMR (400 MHz, DMSO-d₆) δ 4 (s, 3H), 6.75 (dd, J=8.5, 2 Hz, 1H), 6.81 (s, 1H), 6.85 (d, J=2 Hz, 1H), 7.09-7.30 (m, 1H), 7.45-7.62 (m, 2H), 7.67 (d, J=8.5 Hz, 1H), 8.3 (d, J=8.7 Hz, 1H), 8.42 (d, J=8.7 Hz, 1H), 11.38 ppm (s, 1H); ¹³C NMR (101 MHz, DMSO-d₆) δ 55.75, 98.59, 109.12, 110.09, 112.2, 113.21, 119.05, 122.67, 125.9, 127.71, 127.77, 136.19, 139.81, 149.11, 150.13, 155.13, 166.87, 168.12, 181.22 ppm; MS (ACPI) m/z 320.0 (MH⁺, 100).

### (2Z)-6-Hydroxy-2-(quinolin-4-ylmethylene)-1-benzofuran-3(2H)-one (3h)

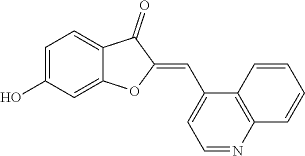 [SMILES: O=C1/C(=C/c2ccnc3ccccc23)Oc2cc(O)ccc21]

Yellow crystals (64% yield); mp 278-280° C.; ¹H NMR (400 MHz, DMSO-d₆) δ 6.76 (dd, J=8.5, 2 Hz, 1H), 6.81 (d, J=2 Hz, 1H), 7.39 (s, 1H), 7.61-7.75 (m, 2H), 7.78-7.91 (m, 1H), 8.09 (dd, J=8.4, 1.3 Hz, 1H), 8.15 (d, J=4.6 Hz, 1H), 8.34 (dd, J=8.5, 1.4 Hz, 1H), 9.03 (d, J=4.6 Hz, 1H), 11.4 ppm (s, 1H); ¹³C NMR (101 MHz, DMSO-d₆) δ 98.54, 102.42, 112.17, 113.13, 121.61, 123.19, 125.5, 125.84, 126.86, 129.12, 129.49, 135.85, 147.94, 149.72, 150.05, 166.79, 168.06, 180.62 ppm; MS (ACPI) m/z 290.0 (MH⁺, 100).

### (2Z)-6-Hydroxy-2-(1H-indol-3-ylmethylene)-1-benzofuran-3(2H)-one (3i)

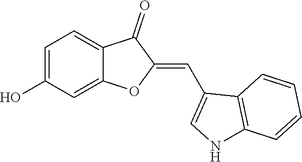 [SMILES: O=C1/C(=C/c2c[nH]c3ccccc23)Oc2cc(O)ccc21]

Yellow crystals (55% yield); mp 280-282° C.; ¹H NMR (400 MHz, DMSO-d₆) δ 6.72 (dd, J=8.5, 2 Hz, 1H), 6.85 (d, J=2 Hz, 1H), 7.11-7.28 (m, 3H), 7.51 (d, J=7.8 Hz, 1H), 7.62 (d, J=8.5 Hz, 1H), 8.01 (d, J=7.7 Hz, 1H), 8.21 (d, J=2.8 Hz, 1H), 10.99 (s, 1H), 11.99 ppm (s, 1H); ¹³C NMR (125 MHz, DMSO-d₆) δ 98.75, 105.36, 108.49, 112.4, 112.76, 114.45, 118.98, 120.86, 122.74, 125.52, 126.9, 131.31, 136.43, 145.43, 165.79, 166.9, 180.52 ppm; MS (ACPI) m/z 278.2 (MH⁺, 100).

### (2Z)-6-Hydroxy-2-[(1-methyl-1H-indol-3-yl)methylene]-1-benzofuran-3(2H)-one (3j)

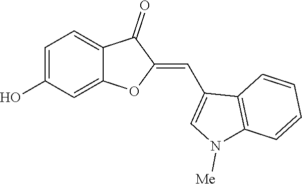 [SMILES: Cn1cc(/C=C2\Oc3cc(O)ccc3C2=O)c2ccccc21]

Yellow crystals (78% yield); mp 272-274° C.; ¹H NMR (400 MHz, DMSO-d₆) δ 3.9 (s, 3H), 6.72 (d, J=8.4 Hz, 1H), 6.83 (s, 1H), 7.13-7.32 (m, 3H), 7.51 (d, J=8 Hz, 1H), 7.6 (d, J=8.1 Hz, 1H), 7.99 (d, J=7.8 Hz, 1H), 8.19 (s, 1H), 11.02 ppm (s, 1H); ¹³C NMR (126 MHz, DMSO-d₆) δ 33.13, 98.48, 104.67, 107.3, 110.54, 112.6, 114.26, 118.96, 120.96, 122.64, 125.41, 127.2, 134.74, 136.76, 145.12, 165.57, 166.65, 180.17 ppm; MS (ACPI) m/z 292.0 (MH⁺, 100).

### (2Z)-6-Hydroxy-2-[(5-methoxy-1-methyl-1H-indol-3-yl)methylene]-1-benzofuran-3(2H)-one (3k)

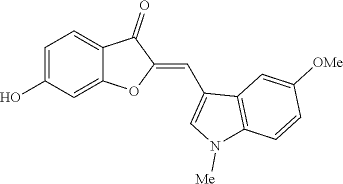 [SMILES: COc1ccc2c(c1)c(/C=C1\Oc3cc(O)ccc3C1=O)cn2C]

Yellow crystals (82% yield); mp 301-303° C.; ¹H NMR (400 MHz, DMSO-d₆) δ 3.84 (s, 3H), 3.86 (s, 3H), 6.71 (dd, J=8.5, 2 Hz, 1H), 6.82 (d, J=2 Hz, 1H), 6.88 (dd, J=8.7, 2.4 Hz, 1H), 7.21 (s, 1H), 7.39 (d, J=8.8 Hz, 1H), 7.53-7.64 (m, 2H), 8.12 (s, 1H), 11.03 ppm (s, 1H); ¹³C NMR (126 MHz, DMSO-d₆) δ 33.28, 55.45, 98.41, 100.92, 105.32, 107.19, 111.38, 112.54, 112.73, 114.33, 125.32, 127.99, 131.82, 134.99, 144.74, 155.05, 165.48, 166.48, 180.07 ppm; MS (ACPI) m/z 322.0 (MH⁺, 100).

### (2Z)-2-[(1-Ethyl-5-methoxy-1H-indol-3-yl)methylene]-6-hydroxy-1-benzofuran-3(2H)-one (3l)

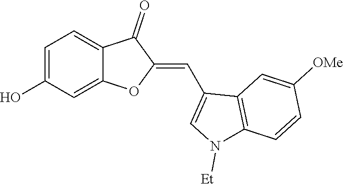 [SMILES: CCn1cc(/C=C2\Oc3cc(O)ccc3C2=O)c2cc(OC)ccc21]

Yellow crystals (77% yield); mp 265-267° C.; ¹H NMR (400 MHz, DMSO-d₆); δ 1.39 (t, J=7.2 Hz, 3H), 3.85 (s, 3H), 4.27 (q, J=7.2 Hz, 2H), 6.72 (dd, J=8.4, 2 Hz, 1H), 6.83 (d, J=2 Hz, 1H), 6.87 (dd, J=8.9, 2.4 Hz, 1H), 7.23 (s, 1H), 7.42 (d, J=8.8 Hz, 1H), 7.56-7.64 (m, 2H), 8.18 (s, 1H), 10.98 ppm (s, 1H); ¹³C NMR (126 MHz, DMSO-d₆); δ 15.38, 41.3, 55.46, 98.49, 101.1, 105.37, 107.42, 111.41, 112.56, 112.76, 114.38, 125.36, 128.22, 130.74, 133.5, 144.76, 155.02, 165.47, 166.52, 180.12 ppm; MS (ACPI) m/z 336.0 (MH⁺, 100).

### (2Z)-2-[(1-Ethyl-1H-indol-3-yl)methylene]-6-hydroxy-1-benzofuran-3(2H)-one (3m)

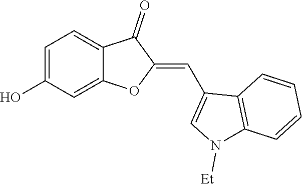 [SMILES: CCn1cc(/C=C2\Oc3cc(O)ccc3C2=O)c2ccccc21]

Yellow crystals (79% yield); mp 278-280° C.; ¹H NMR (400 MHz, DMSO-d₆) δ 1.39 (d, J=7.2 Hz, 3H), 4.3 (q, J=7.2 Hz, 2H), 6.74 (dd, J=8.4, 2.1 Hz, 1H), 6.87 (d, J=2.1 Hz, 1H), 7.11-7.35 (m, 3H), 7.52 (d, J=8 Hz, 1H), 7.63 (d, J=8.4 Hz, 1H), 7.99 (d, J=7.7 Hz, 1H), 8.25 (s, 1H), 11.02 ppm (s, 1H); ¹³C NMR (125 MHz, DMSO-d₆) δ 15.29, 41.14, 98.57, 104.65, 107.54, 110.54, 112.62, 114.29, 119.13, 120.93, 122.62, 125.4, 127.42, 133.22, 135.71, 145.16, 165.61, 166.79, 180.22 ppm; MS (ACPI) m/z 306.1 (MH⁺, 100).

### (2Z)-6-Hydroxy-7-methyl-2-[(1-methyl-1H-indol-3-yl)methylene]-1-benzofuran-3(2H)-one (3n)

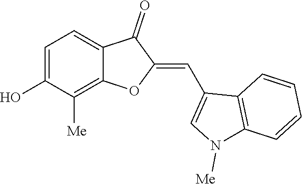 [SMILES: Cc1c(O)ccc2c1O/C(=C\c1cn(C)c3ccccc13)C2=O]

Yellow crystals (89% yield); mp 293-295° C.; ¹H NMR (400 MHz, DMSO-d₆) δ 2.29 (s, 3H), 3.91 (s, 3H), 6.76 (d, J=8.4 Hz, 1H), 7.13 (s, 1H), 7.18-7.32 (m, 2H), 7.43 (d, J=8.4 Hz, 1H), 7.51 (d, J=8.1 Hz, 1H), 8.03 (d, J=7.9 Hz, 1H), 8.07 (s, 1H), 10.83 ppm (s, 1H); ¹³C NMR (125 MHz, DMSO-d₆) δ 7.89, 33.15, 104.34, 107.5, 107.52, 110.51, 111.6, 113.86, 119.08, 120.84, 122.06, 122.61, 127.07, 134.58, 136.79, 145.24, 163.11, 164.81, 180.77 ppm; MS (ACPI) m/z 306.1 (MH⁺, 100).

### (2Z)-6-Hydroxy-2-[(5-methoxy-1-methyl-1H-indol-3-yl)methylene]-7-methyl-1-benzofuran-3(2H)-one (3o)

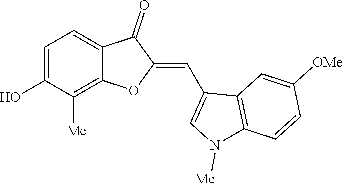 [SMILES: COc1ccc2c(c1)c(/C=C1\Oc3c(ccc(O)c3C)C1=O)cn2C]

Yellow crystals (83% yield); mp 297-299° C.; ¹H NMR (400 MHz, DMSO-d₆) δ 2.28 (s, 3H), 3.84 (s, 3H), 3.88 (s, 3H), 6.75 (d, J=8.3 Hz, 1H), 6.88 (dd, J=8.8, 2.4 Hz, 1H), 7.19 (s, 1H), 7.31-7.49 (m, 2H), 7.56 (d, J=2.4 Hz, 1H), 8.02 (s, 1H), 10.8 ppm (s, 1H); ¹³C NMR (126 MHz, DMSO-d₆) δ 7.89, 33.33, 55.53, 100.96, 104.89, 107.29, 107.5, 111.36, 111.55, 112.68, 113.97, 122, 127.98, 131.8, 134.72, 144.97, 155.02, 162.98, 164.71, 180.69 ppm; MS (ACPI) m/z 336.1 (MH⁺, 100).

### (Z)-2-((2-((1-Ethyl-5-methoxy-1H-indol-3-yl)methylene)-3-oxo-2,3-dihydrobenzofuran-6-yl)oxy)acetonitrile (4a)

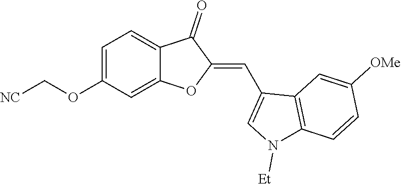 [SMILES: CCn1cc(/C=C2\Oc3cc(OCC#N)ccc3C2=O)c2cc(OC)ccc21]

To a solution of 670 mg (2 mmol) of (2Z)-2-[(1-ethyl-5-methoxy-1H-indol-3-yl)methylene]-6-hydroxy-1-benzofuran-3(21-1)-one (30 in 10 mL of N,N-dimethylformamide was added 830 mg (6 mmol, 3 eq) of anhydrous potassium carbonate. The mixture was heated to 60° C. and 0.152 mL (2.4 mmol, 1.2 eq) of chloroacetonitrile was added. The mixture was stirred at 60° C. for an additional 8 h, cooled, and poured into 100 mL of 0.1N aqueous sulfuric acid. The precipitate was collected by filtration, washed with water, dried and re-crystallized from N,N-dimethylformamide-methanol to afford 487 mg (65%) of 4a as yellow crystals: mp 230-232° C.; ¹H NMR (400 MHz, DMSO-d₆) δ 1.44 (d, J=7.2 Hz, 3H), 3.86 (s, 3H), 4.33 (q, J=7.2 Hz, 2H), 5.39 (s, 2H), 6.9 (dd, J=8.9, 2.4 Hz, 1H), 6.97 (dd, J=8.6, 2.2 Hz, 1H), 7.29 (d, J=2.2 Hz, 1H), 7.37 (s, 1H), 7.51 (d, J=8.9 Hz, 1H), 7.63 (d, J=2.4 Hz, 1H), 7.77 (d, J=8.6 Hz, 1H), 8.23 ppm (s, 1H); ¹³C NMR (100 MHz, DMSO-d₆) δ 14.67, 40.97, 53.89, 55.34, 98.17, 101.45, 106.18, 107.17, 110.99, 111.68, 112.46, 115.45, 116.87, 124.84, 127.89, 130.8, 133.46, 144.2, 154.96, 162.6, 165.49, 179.58 ppm; MS (ACPI) m/z 375.2 (MH⁺, 100).

### 5-Benzyloxy-1-ethyl-1H-indole-3-carboxaldehyde

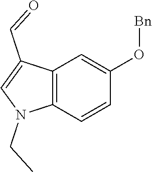 [SMILES: CCn1cc(C=O)c2cc(OCc3ccccc3)ccc21]

To 37 mg (0.14 mmol, 1 eq) 5-benzyloxy-1H-indole-3-carboxaldehyde (Sigma-Aldrich, St. Louis, Mo.) in 0.3 mL of N,N-dimethylformamide under an argon atmosphere was added 5.7 mg (0.196 mmol, 1.4 eq) of 60% sodium hydride in mineral oil. The mixture was stirred for 40 min, and 17 μL (0.21 mmol, 1.5 eq) of ethyl iodide was added dropwise. The reaction mixture was stirred at 25° C. for 12 h. The product was diluted with dichloromethane, washed successively with brine and water, dried, and chromatographed on silica gusing 1:1 ethyl acetate-hexane to afford 31 mg (76%) of 5-benzyloxy-1-ethyl-1H-indole-3-carboxaldehyde: ¹H NMR (400 MHz, CDCl₃) δ 9.96 (s, 1H), 7.92 (d, J=2.5 Hz, 1H), 7.69 (s, 1H), 7.54-7.47 (m, 2H), 7.44-7.37 (m, 2H), 7.35-7.3 (m, 1H), 7.26 (d, J=5 Hz, 1H), 7.05 (dd, J=2.5, 8.9 Hz, 1H), 5.15 (s, 2H), 4.2 (q, J=7.3 Hz, 2H), 1.54 (t, J=7.3 Hz, 3H). ¹³C NMR (101 MHz, CDCl₃) δ 184.36, 155.81, 137.49, 137.21, 132.07, 128.52, 127.88, 126.24, 117.95, 114.97, 110.79, 104.80, 70.62, 42.04, 15.09.

### 5-(tert-Butyldimethylsilyloxy)-1-ethyl-1H-indole-3-carboxaldehyde

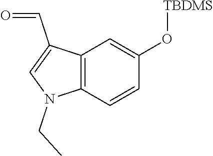

To 14.5 mg (0.05 mmol, 1 eq) of 5-benzyloxy-1-ethyl-1H-indole-3-carboxaldehyde, 22 mg (0.3 mmol, 3 eq) of pentamethylbenzene, and 0.4 mL of anhydrous DMF (0.4 mL) under argon atmosphere at −78° C. was added dropwise 300 μL (0.3 mmol, 6 eq) of boron trichloride in 1M dichloromethane. After stirring for 1 h, the reaction was quenched with 4 mL of saturated aqueous ammonium chloride solution. The mixture was extracted with dichloromethane. The organic solutions were washed successively with brine and water, dried and concentrated to afford a crude product that was used directly in the next reaction. To the crude product was added 9 mg (0.06 mmol, 1.2 eq) of tert-butyldimethylsilyl chloride and 20.4 mg (0.15 mmol, 3 eq) of imidazole in 0.4 mL of anhydrous N,N-dimethylformamide. The mixture was stirred for 2 h. The product was extracted with dichloromethane, washed successively with brine solution and water, and dried. The product was chromatographed on silica gel using 1:1 ethyl acetate-hexane to afford 10 mg (63%) of 5-(tert-butyldimethylsilyloxy)-1-ethyl-1H-indole-3-carbaldehyde: ¹H NMR (400 MHz, CDCl₃) δ 9.94 (s, 1H), 7.75 (d, J=2.3 Hz, 1H), 7.68 (s, 1H), 7.21 (d, J=8.8 Hz, 1H), 6.87 (dd, J=2.4, 8.8 Hz, 1H), 4.18 (q, J=7.3 Hz, 2H), 1.54 (t, J=7.3 Hz, 3H), 1.01 (s, 9H), 0.22 (s, 6H). ¹³C NMR (101 MHz, CDCl₃) δ 188.58, 156.51, 141.95, 136.86, 130.9, 122.25, 122.18, 116.26, 114.72, 46.39, 30.18, 22.64, 19.43, 4.48.

### 2-((3-Oxo-2,3-dihydrobenzofuran-6-yl)oxy)acetonitrile

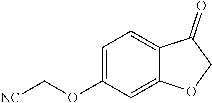 [SMILES: N#CCOc1ccc2c(c1)OCC2=O]

To a solution of 33.5 μL (1.2 eq) of bromoacetonitrile and 60 mg (0.4 mmol, 1 eq) of 6-hydroxybenzofuran-3(2H)-one (1) in 1 mL of acetonitrile was added 110 mg (0.8 mmol, 2 eq) of anhydrous potassium carbonate. The mixture was stirred overnight at 25° C. The product was purified by column chromatography on silica gel 60 with an elution gradient of 1:5 to 1:1 ethyl acetate-hexane to provide 45 mg (60%) of 2-((3-oxo-2,3-dihydrobenzofuran-6-yl)oxy)acetonitrile: ¹H NMR (400 MHz, CDCl₃) δ 4.67 (s, 2H), 4.86 (s, 2H), 6.67 (d, J=2.2 Hz, 1H), 6.73 (dd, J=8.6, 2.2 Hz, 1H), 7.65 ppm (d, J=8.6 Hz, 1H); ¹³C NMR (100 MHz, CDCl₃) δ=53.52, 75.82, 98.06, 111.31, 114.15, 116.52, 125.97, 164.33, 175.99, 197.62 ppm.

### (Z)-2-((2-((1-Ethyl-5-hydroxy-1H-indol-3-yl)methylene)-3-oxo-2,3-dihydrobenzofuran-6-yl)oxy)acetonitrile (4b)

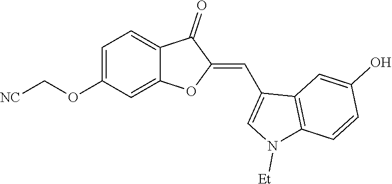 [SMILES: CCn1cc(/C=C2\Oc3cc(OCC#N)ccc3C2=O)c2cc(O)ccc21]

To 36 mg (0.11 mmol, 1 eq) of 5-(tert-butyldimethylsilyloxy)-1-ethyl-1H-indole-3-carboxaldehyde in 2 mL of anhydrous dichloromethane was added 30 mg (0.16 mmol, 1.4 eq) of 2-((3-oxo-2,3-dihydrobenzofuran-6-yl)oxy)acetonitrile and 366 mg (3.6 mmol, 32 eq) of alumina (Sigma-Aldrich, St. Louis, Mo.). The mixture was stirred at 25° C. for 6 h, filtered and concentrated. The residue was treated with 824 μL of a 1M (2.5 eq) solution of tetra(n-butyl)ammonium fluoride in tetrahydrofuran for 1 h at 25° C. The product was recrystallized from ca. 1:1 methanol-dichloromethane to afford 24 mg (60%) of 4b as yellow crystals: mp 208-210° C.; ¹H NMR (400 MHz, DMSO-d₆) δ 1.43 (t, J=7.2 Hz, 4H), 4.29 (q, J=7.2 Hz, 2H), 5.38 (s, 2H), 6.8 (dd, J=8.8, 2.2 Hz, 1H), 6.96 (dd, J=8.6, 2.1 Hz, 1H), 7.11 (s, 1H), 7.3 (dd, J=6.4, 2.1 Hz, 2H), 7.76 (d, J=8.5 Hz, 1H), 8.17 (s, 1H), 9.16 ppm (s, 1H); ¹³C NMR (100 MHz, DMSO-d₆) δ 15.35, 41.35, 54.05, 98.26, 103.37, 106.73, 106.76, 111.45, 112.11, 112.83, 116.19, 117.02, 125.35, 128.45, 134.08, 144.17, 152.8, 162.79, 165.73, 179.84 ppm.

### tert-Butyl 2-(3-formyl-5-methoxy-1H-indol-1-yl)acetate

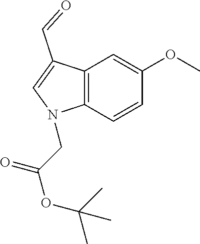 [SMILES: COc1ccc2c(c1)c(C=O)cn2CC(=O)OC(C)(C)C]

To 105 mg (0.6 mmol, 1 eq) of 5-methoxy-1H-indole-3-carboxaldehyde (VWR, Chicago, Ill.) in 1 mL of anhydrous N,N-dimethylformamide under an argon atmosphere was added 32 mg (0.8 mmol, 1.33 eq) of 60% sodium hydride in mineral oil (60 wt %). The mixture was stirred for 40 min at 0° C. To this solution was added dropwise 104 μL (0.7 mmol, 1.16 eq) of tert-butyl bromoacetate (Sigma-Aldrich, St. Louis, Mo.). The mixture was stirred at 25° C. for 12 h, and the solution was extracted with ethyl acetate. The combined organic solutions were washed successively with brine and water and dried. The product was purified by column chromatography on silica gel 60 with gradient elution by 1:5 and 1:2 ethyl acetate-hexane to provide 119 mg (69%) of tert-butyl 2-(3-formyl-5-methoxy-1H-indol-1-yl)acetate: mp 92-94° C. ¹H NMR (400 MHz, DMSO-d₆) δ 9.89 (s, 1H), 8.20 (s, 1H), 7.60 (d, J=2.5 Hz, 1H), 7.41 (d, J=8.9 Hz, 1H), 6.93 (dd, J=2.5, 8.9 Hz, 1H), 5.14 (s, 2H), 3.79 (s, 3H), 1.42 (s, 9H). ¹³C NMR (101 MHz, DMSO-d₆) δ 184.76, 167.30, 155.96, 141.74, 132.46, 125.19, 117.34, 113.31, 111.84, 102.70, 82.01, 55.37, 48.36, 27.68.

### (Z)-2-(3-((6-(Cyanomethoxy)-3-oxobenzofuran-2(3H)-ylidene)methyl)-5-methoxy-1H-indol-1-yl)acetic Acid (4c)

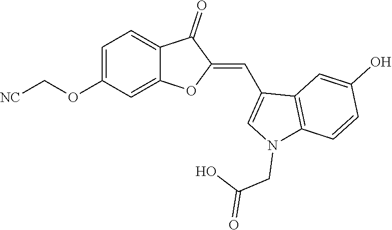 [SMILES: N#CCOc1ccc2c(c1)O/C(=C\c1cn(CC(=O)O)c3ccc(O)cc13)C2=O]

To a solution of 45 mg (0.24 mmol, 1.2 eq) of 2-(3-oxo-2,3-dihydrobenzofuran-6-yl)oxy)acetonitrile in 3 mL of anhydrous dichloromethane was added 58 mg (0.2 mmol, 1 eq) of tert-butyl 2-(3-formyl-5-methoxy-1H-indol-1-yl)acetate and 646 mg (6.4 mmol, 32 eq) of alumina. The mixture was stirred at 25° C. for 6 h, filtered, concentrated and recrystallized from ca. 1:4 methanol-dichloromethane, to afford pure tert-butyl (Z)-2-(3-((6-(cyanomethoxy)-3-oxobenzofuran-2(3H)-ylidene)methyl)-5-methoxy-1H-indol-1-yl)acetate. This product was refluxed in 2 mL of formic acid 60° C. for 2 h. The solution was concentrated to afford, 48 mg (60%) of 4c as yellow crystals: mp>220° C.; NMR (400 MHz, DMSO-d₆) δ 3.85 (s, 3H), 5.18 (s, 2H), 5.38 (s, 2H), 6.88 (dd, J=8.9, 2.4 Hz, 1H), 6.97 (dd, J=8.5, 2.2 Hz, 1H), 7.23 (d, J=2.1 Hz, 1H), 7.37 (s, 1H), 7.43 (d, J=8.9 Hz, 1H), 7.63 (d, J=2.4 Hz, 1H), 7.78 (d, J=8.5 Hz, 1H), 8.22 (s, 1H), 13.18 ppm (s, 1H); ¹³C NMR (101 MHz, DMSO-d₆) δ 54.04, 55.56, 98.11, 100.99, 106.62, 107.83, 111.63, 112.24, 113.01, 116.17, 116.94, 125.52, 128.02, 131.69, 135.55, 144.83, 155.23, 162.94, 165.92, 169.99, 180.10 ppm; MS (ACPI) m/z 391.2 (MH⁺, 100).

General Procedure for the Synthesis of Aurones 4d-4q.

To a solution of 2 mmol of aurones 3 in 10 mL of N,N-dimethylformamide was added 830 mg (6 mmol) of anhydrous potassium carbonate. The mixture was heated to 60° C. and 2.4 mmol of the appropriate benzyl chloride was added. The mixture was stirred at 60° C. for 8 h, cooled, and poured into 100 mL of aqueous 0.1N sulfuric acid. The precipitate was collected by filtration, washed with water, dried and re-crystallized from N,N-dimethylformamide-methanol 1:1-1:2.

### (2Z)-6-[(2-Fluorobenzyl)oxy]-2-[(1-methyl-1H-indol-3-yl)methylene]-1-benzofuran-3(2H)-one (4d)

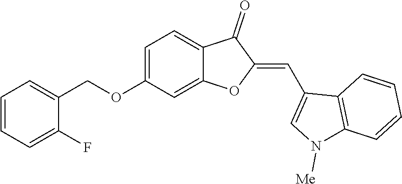 [SMILES: Cn1cc(/C=C2\Oc3cc(OCc4ccccc4F)ccc3C2=O)c2ccccc21]

Yellow crystals (76% yield); mp 241-243° C.; ¹H NMR (400 MHz, DMSO-d₆) δ 3.93 (s, 3H), 5.34 (s, 2H), 6.93 (dd, J=8.4, 2.2 Hz, 1H), 7.14-7.35 (m, 6H), 7.39-7.72 (m, 4H), 8.02 (d, J=7.8 Hz, 1H), 8.15 ppm (s, 1H); ¹³C NMR (125 MHz, CDCl₃) δ 33.47, 64.31 (d, J=4.6 Hz), 97.42, 105.95, 108.57, 109.8, 112.12, 115.52 (d, J=21.1 Hz), 116.62, 119.17, 121.28, 123.01, 124.44 (d, J=3.4 Hz), 125.55, 127.87, 129.61, 129.64, 130.18 (d, J=8.4 Hz), 134.01, 136.96, 146.01, 160.43 (d, J=247.5 Hz), 165.36, 167, 181.75 ppm; MS (ACPI) m/z 400.0 (MH⁺, 100).

### (2Z)-6-[(2-Chlorobenzyl)oxy]-2-[(1-methyl-1H-indol-3-yl)methylene]-1-benzofuran-3(2H)-one (4e)

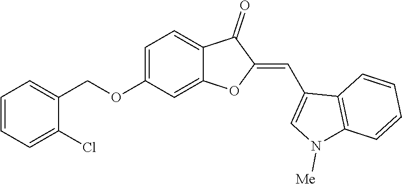 [SMILES: Cn1cc(/C=C2\Oc3cc(OCc4ccccc4Cl)ccc3C2=O)c2ccccc21]

Yellow crystals (69% yield); mp 206-208° C.; ¹H NMR (400 MHz, DMSO-d₆) δ 3.93 (s, 3H), 5.35 (s, 2H), 6.93 (dd, J=8.5, 2.2 Hz, 1H), 7.17-7.36 (m, 4H), 7.38-7.49 (m, 2H), 7.5-7.59 (m, 2H), 7.6-7.73 (m, 2H), 8.04 (d, J=7.7 Hz, 1H), 8.21 ppm (s, 1H); ¹³C NMR (126 MHz, CDCl₃) δ 33.53, 67.78, 97.54, 106.03, 108.61, 109.92, 112.24, 116.72, 119.18, 121.36, 123.08, 125.56, 127.24, 127.96, 128.82, 129.51, 129.64, 132.74, 133.67, 134.17, 137.01, 146.08, 165.37, 167.04, 181.78 ppm; MS (ACPI) m/z 416.0 (MH⁺, 100).

### (2Z)-6-[(2,6-Difluorobenzyl)oxy]-2-[(1-methyl-1H-indol-3-yl)methylene]-1-benzofuran-3(2H)-one (4f)

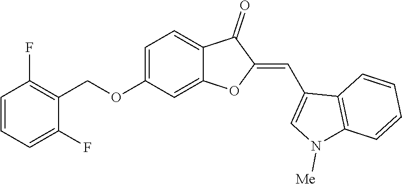 [SMILES: Cn1cc(/C=C2\Oc3cc(OCc4c(F)cccc4F)ccc3C2=O)c2ccccc21]

Yellow crystals (73% yield); mp>220° C.; ¹H NMR (400 MHz, DMSO-d₆) δ 3.94 (s, 3H), 5.31 (s, 2H), 6.9 (dd, J=8.5, 2.1 Hz, 1H), 7.19-7.40 (m, 6H), 7.54-7.61 (m, 2H), 7.69 (d, J=8.5 Hz, 1H), 8.08 (d, J=7.8 Hz, 1H), 8.21 ppm (s, 1H); ¹³C NMR (101 MHz, DMSO-d₆) δ 33.67, 58.99, 98.11, 106.19, 107.8, 111.09, 111.95 (t, J=18.7 Hz), 112.36 (d, J=24.2 Hz), 112.88, 116.21, 119.65, 121.54, 123.22, 125.56, 127.5, 132.56 (t, J=10.2 Hz), 135.55, 137.28, 145.29, 161.63 (dd, J=256.8, 7.1 Hz), 165.48, 166.82, 180.58 ppm; MS (ACPI) m/z 418.2 (MH⁺, 100).

### (2Z)-6-[(2,6-Dichlorobenzyl)oxy]-2-[(1-methyl-1H-indol-3-yl)methylene]-1-benzofuran-3(2H)-one (4g)

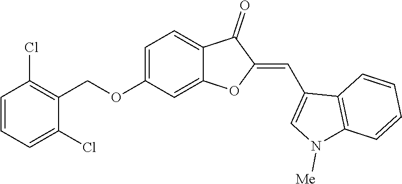 [SMILES: Cn1cc(/C=C2\Oc3cc(OCc4c(Cl)cccc4Cl)ccc3C2=O)c2ccccc21]

Yellow crystals (65% yield); mp 247-249° C.; ¹H NMR (400 MHz, CDCl₃) δ 3.9 (s, 3H), 5.4 (s, 2H), 6.84 (dd, J=8.5, 2.1 Hz, 1H), 6.95 (d, J=2.1 Hz, 1H), 7.18-7.48 (m, 7H), 7.75 (d, J=8.5 Hz, 1H), 7.92 (d, J=7.7 Hz, 1H), 7.97 ppm (s, 1H); ¹³C NMR (1010 MHz, CDCl₃) δ 33.63, 65.82, 97.65, 106.12, 108.75, 109.96, 112.23, 116.81, 119.33, 121.43, 123.16, 125.67, 128.01, 128.72, 131, 131.28, 134.2, 137.1, 137.18, 146.18, 165.83, 167.1, 181.94 ppm; MS (ACPI) m/z 450.0 (MH³⁰, 100).

### (2Z)-6-[(2-Chloro-6-fluorobenzyl)oxy]-2-[(1-methyl-1H-indol-3-yl)methylene]-1-benzofuran-3(2H)-one (4h)

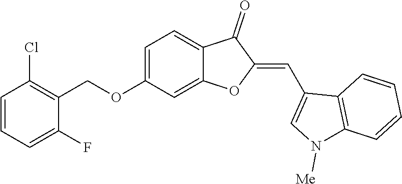 [SMILES: Cn1cc(/C=C2\Oc3cc(OCc4c(F)cccc4Cl)ccc3C2=O)c2ccccc21]

Yellow crystals (76% yield); mp 225-227° C.; ¹H NMR (400 MHz, CDCl₃) δ 3.87 (s, 3H), 5.28 (d, J=1.8 Hz, 2H), 6.8 (dd, J=8.5, 2.1 Hz, 1H), 6.91 (d, J=2.1 Hz, 1H), 7.03-7.13 (m, 1H), 7.24-7.41 (m, 6H), 7.72 (d, J=8.5 Hz, 1H), 7.90 (d, J=7.6 Hz, 1H), 7.94 ppm (s, 1H); ¹³C NMR (126 MHz, CDCl₃) δ 33.59, 61.83 (d, J=4.1 Hz), 97.63, 106.05, 108.73, 109.94, 112.17, 114.57 (d, J=22.5 Hz), 116.79, 119.3, 121.41, 121.61 (d, J=17.4 Hz), 123.14, 125.62, 125.82 (d, J=3.3 Hz), 128, 131.31 (d, J=9.7 Hz), 134.18, 136.74 (d, J=4.6 Hz), 137.09, 146.16, 162.12 (d, J=251.8 Hz), 165.64, 167.07, 181.89 ppm; MS (ACPI) m/z 434.0 (MH⁺, 100).

### (2Z)-6-[(2,6-Dichlorobenzyl)oxy]-7-methyl-2-[(1-methyl-1H-indol-3-yl)methylene]-1-benzofuran-3(2H)-one (4i)

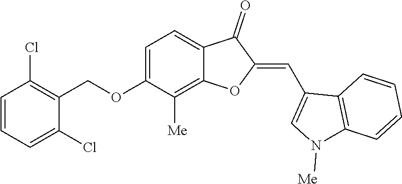 [SMILES: Cc1c(OCc2c(Cl)cccc2Cl)ccc2c1O/C(=C\c1cn(C)c3ccccc13)C2=O]

Yellow crystals (87% yield); mp 232-234° C.; ¹H NMR (400 MHz, DMSO-d₆) δ: 2.25 (s, 3H), 3.93 (s, 3H), 5.40 (s, 2H), 7.15-7.33 (m, 4H), 7.46-7.62 (m, 4H), 7.66 (d, J=8.5 Hz, 1H), 8.05 (d, J=7.9 Hz, 1H), 8.12 ppm (s, 1H); ¹³C NMR (125 MHz, CDCl₃) δ 8.16, 33.57, 66.25, 105.45, 107.75, 108.88, 109.85, 110.91, 116.61, 119.22, 121.23, 122.6, 123.01, 127.89, 128.62, 130.73, 131.71, 134, 136.96, 137.05, 146.24, 163.15, 164.54, 182.64 ppm; MS (ACPI) m/z 464.0 (MH⁺, 100).

### (2Z)-6-[(2-Chloro-4-fluorobenzyl)oxy]-2-[(5-methoxy-1-methyl-1H-indol-3-yl)methylene]-1-benzofuran-3(2H)-one (4j)

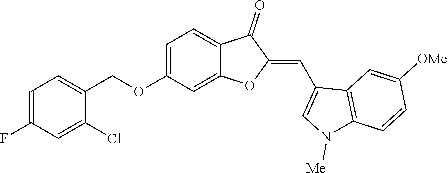 [SMILES: COc1ccc2c(c1)c(/C=C1\Oc3cc(OCc4ccc(F)cc4Cl)ccc3C1=O)cn2C]

Yellow crystals (85% yield); mp 184-186° C.; ¹H NMR (400 MHz, DMSO-d₆) δ 3.85 (s, 3H), 3.89 (s, 3H), 5.3 (s, 2H), 6.85-6.97 (m, 2H), 7.20 (d, J=2.1 Hz, 1H), 7.26-7.37 (m, 2H), 7.45 (d, J=8.9 Hz, 1H), 7.56 (dd, J=8.9, 2.6 Hz, 1H), 7.61 (d, J=2.4 Hz, 1H), 7.65-7.75 (m, 2H), 8.16 ppm (s, 1H); ¹³C NMR (126 MHz, CDCl₃) δ 33.66, 55.84, 67.19, 97.45, 100.58, 106.26, 108.23, 110.78, 111.98, 113.39, 114.44 (d, J=21.1 Hz), 116.8, 117.08 (d, J=24.9 Hz), 125.4, 128.63, 129.66 (d, J=3.6 Hz), 130.14 (d, J=9 Hz), 132.03, 133.55 (d, J=10.5 Hz), 134.43, 145.67, 155.62, 162.34 (d, J=250.5 Hz), 165.03, 166.78, 181.59 ppm; MS (ACPI) m/z 464.0 (MH⁺, 100).

### (2Z)-6-[(2-Chloro-6-fluorobenzyl)oxy]-2-[(5-methoxy-1-methyl-1H-indol-3-yl)methylene]-1-benzofuran-3(2H)-one (4k)

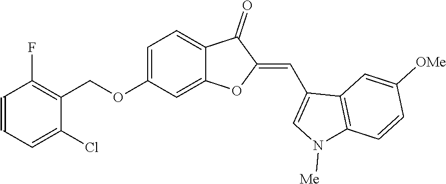 [SMILES: COc1ccc2c(c1)c(/C=C1\Oc3cc(OCc4c(F)cccc4Cl)ccc3C1=O)cn2C]

Yellow crystals (71% yield); mp 200-202° C.; ¹H NMR (400 MHz, DMSO-d₆) δ 3.85 (s, 3H), 3.9 (s, 3H), 5.33 (d, J=1.8 Hz, 2H), 6.86-6.95 (m, 2H), 7.26-7.4 (m, 3H), 7.42-7.49 (m, 2H), 7.51-7.6 (m, 1H), 7.62 (d, J=2.4 Hz, 1H), 7.69 (d, J=8.5 Hz, 1H), 8.15 ppm (s, 1H); ¹³C NMR (126 MHz, CDCl₃) δ 33.56, 55.78, 61.65 (d, J=4.2 Hz), 97.38, 100.51, 106.13, 108.18, 110.73, 111.96, 113.29, 114.43 (d, J=22.6 Hz), 116.63, 121.47 (d, J=17.2 Hz), 125.31, 125.67 (d, J=3.4 Hz), 128.54, 131.18 (d, J=9.9 Hz), 131.95, 134.42, 136.56 (d, J=4.8 Hz), 145.63, 155.52, 161.96 (d, J=251.9 Hz), 165.39, 166.75, 181.59 ppm; MS (ACPI) m/z 464.2 (MH³⁰, 100).

### (2Z)-6-[(2,6-Dichlorobenzyl)oxy]-2-[(5-methoxy-1-methyl-1H-indol-3-yl)methylene]-1-benzofuran-3(2H)-one (4l)

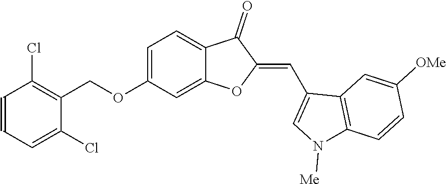 [SMILES: COc1ccc2c(c1)c(/C=C1\Oc3cc(OCc4c(Cl)cccc4Cl)ccc3C1=O)cn2C]

Yellow crystals (69% yield); mp 233-235° C.; ¹H NMR (400 MHz, CDCl₃) δ 3.8 (s, 3H), 3.89 (s, 3H), 5.34 (s, 2H), 6.8 (dd, J=8.5, 2.1 Hz, 1H), 6.89 (d, J=2.1 Hz, 1H), 6.93 (dd, J=8.8, 2.4 Hz, 1H), 7.15-7.41 (m, 6H), 7.71 (d, J=8.5 Hz, 1H), 7.87 ppm (s, 1H); ¹³C NMR (126 MHz, CDCl₃) δ 33.69, 55.9, 65.74, 97.58, 100.66, 106.22, 108.31, 110.8, 112.07, 113.45, 116.79, 125.49, 128.63, 130.91, 131.23, 132.07, 134.43, 137.09, 145.77, 155.63, 165.68, 166.89, 181.71 ppm; MS (ACPI) m/z 480.0 (MH⁺, 100).

### (2Z)-6-[(2,6-Dichlorobenzyl)oxy]-2-[(5-methoxy-1-methyl-1H-indol-3-yl)methylene]-7-methyl-1-benzofuran-3(2H)-one (4m)

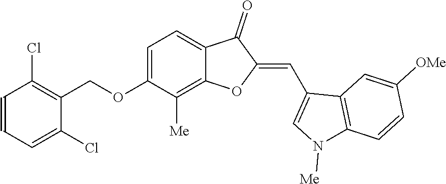 [SMILES: COc1ccc2c(c1)c(/C=C1\Oc3c(ccc(OCc4c(Cl)cccc4Cl)c3C)C1=O)cn2C]

Yellow crystals (74% yield); mp 241-243° C.; ¹H NMR (400 MHz, DMSO-d₆) δ 2.25 (s, 3H), 3.84 (s, 3H), 3.9 (s, 3H), 5.41 (s, 2H), 6.9 (dd, J=8.8, 2.4 Hz, 1H), 7.2 (d, J=8.5 Hz, 1H), 7.29 (s, 1H), 7.41-7.55 (m, 2H), 7.57-7.63 (m, 3H), 7.66 (d, J=8.5 Hz, 1H), 8.08 ppm (s, 1H); ¹³C NMR (126 MHz, CDCl₃) δ=8.21, 33.83, 55.97, 66.32, 100.72, 105.78, 107.83, 108.59, 110.8, 110.96, 113.51, 116.75, 122.66, 128.66, 128.7, 130.76, 131.77, 132.1, 134.27, 137.11, 146.02, 155.62, 163.16, 164.55, 182.67 ppm; MS (ACPI) m/z 494.2 (MH⁺, 100).

### (2Z)-2-[(1-Ethyl-5-methoxy-1H-indol-3-yl)methylene]-6-[(2-fluorobenzyl)oxy]-1-benzofuran-3(2H)-one (4n)

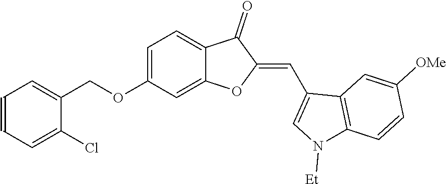 [SMILES: CCn1cc(/C=C2\Oc3cc(OCc4ccccc4Cl)ccc3C2=O)c2cc(OC)ccc21]

Yellow crystals (63% yield); mp 139-141° C.; ¹H NMR (400 MHz, DMSO-d₆) δ 1.42 (t, J=7.2 Hz, 3H), 3.85 (s, 3H), 4.29 (q, J=7.2 Hz, 2H), 5.31 (s, 2H), 6.83-6.94 (m, 2H), 7.18-7.33 (m, 4H), 7.4-7.53 (m, 2H), 7.56-7.64 (m, 2H), 7.67 (d, J=8.5 Hz, 1H), 8.19 ppm (s, 1H); ¹³C NMR (126 MHz, CDCl₃) δ 15.39, 41.93, 55.79, 64.27 (d, J=4.6 Hz), 97.36, 100.63, 106.29, 108.29, 110.81, 112.06, 113.32, 115.48 (d, J=21.1 Hz), 116.63, 123.03 (d, J=14.2 Hz), 124.43 (d, J=3.6 Hz), 125.4, 128.78, 129.61 (d, J=3.6 Hz), 130.15 (d, J=8.2 Hz), 130.97, 132.69, 145.6, 155.48, 160.41 (d, J=247.2 Hz), 165.25, 166.82, 181.64 ppm; MS (ACPI) m/z 444.2 (MH³⁰, 100).

### (2Z)-6-[(2,6-Dichlorobenzyl)oxy]-2-[(1-ethyl-5-methoxy-1H-indol-3-yl)methylene]-1-benzofuran-3(2H)-one (4o)

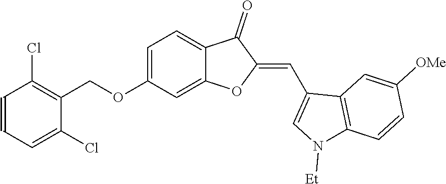 [SMILES: CCn1cc(/C=C2\Oc3cc(OCc4c(Cl)cccc4Cl)ccc3C2=O)c2cc(OC)ccc21]

Yellow crystals (73% yield); mp 211-213° C.; ¹H NMR (400 MHz, CDCl₃) δ 1.54 (t, J=7.2 Hz, 3H), 3.9 (s, 3H), 4.2 (q, J=7.2 Hz, 2H), 5.36 (s, 2H), 6.82 (dd, J=8.6, 2.1 Hz, 1H), 6.89-6.97 (m, 2H), 7.2-7.42 (m, 6H), 7.73 (d, J=8.5 Hz, 1H), 7.97 ppm (s, 1H); ¹³C NMR (126 MHz, CDCl₃) δ 15.46, 42.02, 55.91, 65.75, 97.62, 100.76, 106.33, 108.42, 110.91, 112.09, 113.41, 116.82, 125.51, 128.63, 128.9, 130.91, 131.09, 131.22, 132.76, 137.09, 145.75, 155.59, 165.68, 166.89, 181.74 ppm; MS (ACPI) m/z 494.2 (MH⁺, 100).

### (2Z)-6-[(2-Chloro-6-fluorobenzyl)oxy]-2-[(1-ethyl-1H-indol-3-yl)methylene]-1-benzofuran-3(2H)-one (4p)

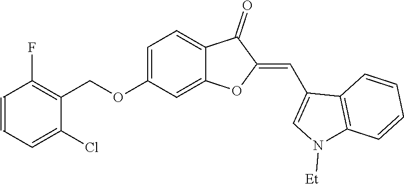 [SMILES: CCn1cc(/C=C2\Oc3cc(OCc4c(F)cccc4Cl)ccc3C2=O)c2ccccc21]

Yellow crystals (74% yield); mp 198-200° C.; ¹H NMR (400 MHz, DMSO-d₆) δ 1.46 (t, J=7.2 Hz, 3H), 4.36 (q, J=7.2 Hz, 2H), 5.35 (d, J=1.8 Hz, 2H), 6.91 (dd, J=8.5, 2.1 Hz, 1H), 7.21-7.33 (m, 3H), 7.33-7.41 (m, 2H), 7.47 (d, J=8.1 Hz, 1H), 7.52-7.59 (m, 1H), 7.61 (d, J=8.1 Hz, 1H), 7.7 (d, J=8.5 Hz, 1H), 8.08 (d, J=7.8 Hz, 1H), 8.28 ppm (s, 1H); ¹³C NMR (126 MHz, CDCl₃) δ 15.31, 41.73, 61.68, 97.46, 105.98, 108.61, 109.95, 112.02, 114.42 (d, J=22.7 Hz), 116.6, 119.18, 121.22, 121.44 (d, J=17.2 Hz), 122.88, 125.36, 125.66, 128.06, 131.18 (d, J=9.8 Hz), 132.51, 135.96, 136.55 (d, J=4.8 Hz), 145.95, 161.95 (d, J=252.5 Hz), 165.46, 166.87, 181.69 ppm; MS (ACPI) m/z 448.2 (MH³⁰, 100).

### (2Z)-6-[(2,6-Dichlorobenzyl)oxy]-2-[(1-ethyl-1H-indol-3-yl)methylene]-1-benzofuran-3(2H)-one (4q)

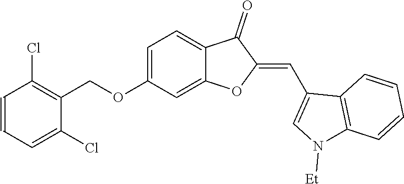 [SMILES: CCn1cc(/C=C2\Oc3cc(OCc4c(Cl)cccc4Cl)ccc3C2=O)c2ccccc21]

Yellow crystals (68% yield); mp 213-215° C.; ¹H NMR (400 MHz, DMSO-d₆) δ 1.46 (t, J=7.2 Hz, 3H), 3.35 (s, 3H), 4.37 (q, J=7.2 Hz, 2H), 5.43 (s, 2H), 6.92 (dd, J=8.5, 2.2 Hz, 1H), 7.18-7.34 (m, 3H), 7.40 (d, J=2.2 Hz, 1H), 7.52 (dd, J=8.9, 7.2 Hz, 1H), 7.59-7.65 (m, 3H), 7.71 (d, J=8.5 Hz, 1H), 8.05-8.13 (m, 1H), 8.29 ppm (s, 1H); ¹³C NMR (126 MHz, CDCl₃) δ 15.43, 41.85, 65.77, 97.6, 106.13, 108.76, 110.03, 112.17, 116.75, 119.35, 121.33, 122.99, 125.55, 128.17, 128.66, 130.93, 131.22, 132.58, 136.08, 137.11, 146.07, 165.75, 167, 181.83 ppm; MS (ACPI) m/z 464.2 (MH⁺, 100).

### (2Z)-6-[(2,6-Dichlorobenzyl)oxy]-2-(pyridin-4-ylmethylene)-1-benzofuran-3(2H)-one (4r)

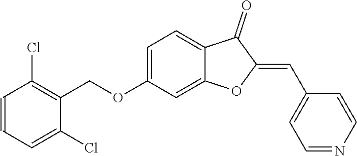 [SMILES: O=C1/C(=C/c2ccncc2)Oc2cc(OCc3c(Cl)cccc3Cl)ccc21]

To a solution of 1.5 g (10 mmol) of 6-hydroxybenzofuran-3(2H)-one (1) in 30 mL of N,N-dimethylformamide was added 4.14 g (30 mmol, 3 eq) of anhydrous potassium carbonate followed by 2.35 g (12 mmol, 1.2 eq) of 2,6-dichlorobenzylchloride (Acros Organics). The mixture was stirred at 25° C. for 8 h and diluted with 200 mL of water. The precipitate was collected, washed with water, dried and purified by column chromatography using 1:100 dichloromethane-methanol to afford 1.79 g (58%) of 6-((2,6-dichlorobenzyl)oxy)benzofuran-3(2H)-one as pale yellow crystals: mp 153-155° C. ¹H NMR (400 MHz, CDCl₃) δ 4.64 (s, 2H), 5.34 (s, 2H), 6.67-6.77 (m, 2H), 7.29 (d, J=7.2 Hz, 1H), 7.33-7.42 (m, 2H), 7.58 (d, J=9 Hz, 1H); ¹³C NMR (100 MHz, CDCl₃) δ 65.57, 75.56, 97.32, 111.98, 114.76, 125.15, 128.56, 130.9, 130.96, 136.97, 167.18, 176.32, 197.49 ppm; MS (ACPI) m/z 309.2 (MH⁺, 100). To 50 mL of a freshly prepared 0.2 M (5 eq) solution of sodium methoxide was added a solution of 618 mg (2 mmol) of 64(2,6-dichlorobenzyl)oxy)benzofuran-3(2H)-one and 214 mg (2 mmol, 1 eq) of 4-pyridinecarboxaldehyde in 5 mL of methanol. The mixture was stirred at 25° C. for 12 h. The solution was concentrated and poured into 100 mL of cold water. The mixture was acidified with 1N aqueous hydrochloric acid solution to ca. pH 6. The precipitate was collected by filtration and recrystallized from 2:1 N,N-dimethylformamide-methanol to afford 445 mg (56%) of 4r: mp 219-222° C.; ¹H NMR (400 MHz, CDCl₃) δ 5.41 (s, 2H), 6.7 (s, 1H), 6.88 (dd, J=8.6, 2.2 Hz, 1H), 6.96 (d, J=2.2 Hz, 1H), 7.28-7.36 (m, 1H), 7.36-7.45 (m, 2H), 7.68-7.78 (m, 3H), 8.7 ppm (d, J=5.2 Hz, 2H); ¹³C NMR (126 MHz, CDCl₃) δ 66.04, 98, 108.3, 113.23, 114.8, 124.74, 126.45, 128.78, 130.92, 131.17, 137.19, 139.95, 150.3, 150.36, 167.19, 168.85, 182.73 ppm; MS (ACPI) m/z 398.0 (MH⁺, 100).

### (2Z)-6-[(2-Chlorobenzyl)oxy]-2-[2-chloro-4-(dimethylamino)benzylidene]-1-benzofuran-3(2H)-one (4s)

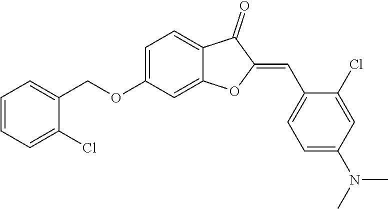 [SMILES: CN(C)c1ccc(/C=C2\Oc3cc(OCc4ccccc4Cl)ccc3C2=O)c(Cl)c1]

Yellow crystals (71% yield); mp>220° C.; ¹H NMR (400 MHz, DMSO-d₆) δ 4.15 (s, 6H), 6.16 (s, 2H), 7.61-7.7 (m, 2H), 7.77 (dd, J=8.6, 2.1 Hz, 1H), 7.85 (s, 1H), 8.08 (d, J=2.1 Hz, 1H), 8.21-8.31 (m, 2H), 8.35-8.41 (m, 1H), 8.45-8.5 (m, 1H), 8.54 (d, J=8.6 Hz, 1H), 9.03 ppm (d, J=8.8 Hz, 1H); ¹³C NMR (101 MHz, DMSO-d₆) δ 39.63, 68.01, 107.11, 111.25, 111.95, 112.85, 114.66, 115.69, 125.42, 127.49, 129.53 (2C), 130.34, 130.59, 132.61, 132.97, 133.3, 136.51, 145.56, 151.64, 165.66, 167.09, 180.91 ppm; MS (ACPI) m/z 441.2 (MH⁺, 100).

### (2Z)-6-[(2,5-Difluorobenzyl)oxy]-2-[4-(diethylamino)-2-methoxybenzylidene]-1-benzofuran-3(2H)-one (4t)

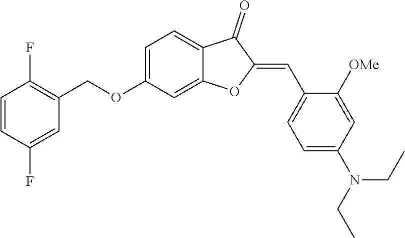 [SMILES: CCN(CC)c1ccc(/C=C2\Oc3cc(OCc4cc(F)ccc4F)ccc3C2=O)c(OC)c1]

Yellow crystals (80% yield); mp 184-186° C.; ¹H NMR (400 MHz, DMSO-d₆) δ 1.15 (t, J=7 Hz, 6H), 3.45 (q, J=7 Hz, 4H), 3.89 (s, 3H), 5.29 (s, 2H), 6.23 (d, J=2.3 Hz, 1H), 6.43 (dd, J=9.1, 2.3 Hz, 1H), 6.92 (dd, J=8.5, 2.1 Hz, 1H), 7.12 (s, 1H), 7.22 (d, J=2.1 Hz, 1H), 7.27-7.4 (m, 2H), 7.44-7.54 (m, 1H), 7.66 (d, J=8.5 Hz, 1H), 8.05 ppm (d, J=9.1 Hz, 1H); ¹³C NMR (100 MHz, DMSO-d₆) δ 12.54, 43.98, 55.5, 64.03, 93.62, 97.81, 104.8, 107.06, 107.33, 112.37, 115.41, 116.69-117.33 (m, 3C), 124.94 (dd, J=17.3, 8.3 Hz), 124.96, 132.69, 144.34, 150.79, 156.45 (d, J=242.8 Hz), 158.05 (d, J=242.7 Hz), 160.51, 164.76, 166.32, 180.42 ppm; MS (ACPI) m/z 466.2 (MH⁺, 100).

Biology.

Cell Culture. LS174T (colon cancer cells), PC-3 (prostate cancer cells), MCF-7 (beast) and A549 (lung) were cultured in the medium recommended by American Type Culture Collection at 37° C. with 5% CO₂ atmosphere in a water jacketed incubator. Ovcar-8 and NCI/ADR-RES are gifts from Dr. Markos Leggas. Alpha-tubulin antibodies, TRITC-conjugated anti-rabbit antibody.

Proliferation inhibition assay. Cancer cells were seeded into 24-well plates at a density of 20,000 cells per well in 1 mL of culture medium and cultured overnight at 37° C. Compounds and the vehicle control DMSO were added to the cells. After 6 days, medium was removed and 100 μL of trypsin was added. Then the cells resuspended in pbs were counted by Vi-CELL XR 2.03 (Beckman Coulter, Inc. USA). The ratio R of the number of viable cells in the compound treatment group to the number of viable cells in DMSO treatment group was taken as relative growth, and the percentage growth inhibition was calculated as: (1−R)*100. For initial testing, compounds were added to the cells at a final concentration 10 μM. Active compounds at 10 μM would be tested at lower concentrations.

In vitro tubulin polymerization assay. The in vitro tubulin polymerization assay was performed by referring tubulin manufacturer's protocol. Briefly, tubulin powder (Cytoskeleton Inc. USA) was dissolved in cold buffer [100 mM PIPES (pH 6.9), 2 mM MgCl₂, 1 mM GTP, and 5% glycerol] on ice. Then aliquots (80 μL, 3.75 μg/μL) of the obtained tubulin solution were divided into the wells of a 96-well half-area plate (Corning, USA). After adding DMSO or testing compounds, the plate was mounted to a Spectra MR™ microplate spectrophotometer equipped with a thermal controller allowing to adjust the temperature to 37° C. (DYNEX Technologies Limited, USA). Readings at 350 nm were recorded every 30 s for 1 hour.

In vivo microtubule assembly assay. The amount of insoluble polymerized microtubules and soluble tubulin dimers in cells after exposure to compounds were detected following the method by Blagosklommy et al. (Cancer research 55, 4623-4626 (1995). Cells were seeded in 6-well plate at 50% confluency and cultured overnight. DMSO or compounds were added and the cells were incubated for additional 6 hours. The medium was removed and cells were washed with PBS three times followed by addition of lysis buffer [20 mM Tris-HCl (pH 6.8), 1 mM MgCl₂, 2 mM EGTA, 20 μg/mL aprotinin, 20 μg/mL leupeptin, 1 mM PMSF, 1 mM orthovanadate, and 0.5% NP40]. The lysates were centrifuged at 12000 g for 10 min to obtain supernatants and pellets, where were mixed with loading buffer and boiled. Then standard western blot analysis against β-tubulin was performed as described by our previous work. (ACS Chem Biol 8, 796-803, doi:10.1021/cb3005353 (2013)).

Immunofluorescence Imaging.

Tubulin networks were examined by confocal immunofluorescence imaging. Briefly, PC3 cells were placed at a density of 80,000/mL to 24-well plates equipped with round microscope glass cover slides. After culturing at 37° C. for 24 hours, DMSO or compounds were added to the cells and incubated for additional 6 hours. Then the medium was removed and the cells were washed with pbs three times. Primary anti-α-tubulin antibody was added and incubated overnight at 4° C. After additional washing, secondary TRITC-conjugated anti-rabbit antibody was added for 40 min, followed by additional washing and staining with DAPI. Final washing was performed and the cover slides were inverted onto glass slides. Images (40×) were taken using a Nikon confocal microscope with excitation at 557 nm and emission at 576 nm.

Molecular Docking.

The X-ray crystal structure of αβ-tubulin binding with colchicine (pdb: 402B) was downloaded from RCSB Protein Data Bank and prepared using AutoDockTools-1.5.6 (Molecular Graphics Laboratory, The Scripps Research Institute). Briefly, αβ-tubulin dimer was separated from 402B using PyMOL (Version 1.7.4.5 Edu). Water molecules were removed and polar hydrogens and Kollman charges were added. The docking pocket (colchicine-binding site) was defined as follows: Search space: 18×18×18 Å³; Center_x, y, z=14.815, 9.422, −20.186. The ligand 4a was prepared by Openbabel. Molecular docking of 4a to the colchicine-binding site was executed using AutoDock vina-1.1.2 using the iterated gradient-based local search method with a Broyden-Fletcher-Goldfarb-Shanno (BFGS) method for local optimization. Exhaustiveness was set at 14 and the number of modes was 9. Other parameters were left as default. The docked tubulin-4a complex with lowest binding free energy was analyzed and presented.

In Vivo Evaluation of Anti-Leukemia Activity in the Zebrafish Model

Rag2:Myc-GFP zebrafish at 21 days of age were treated with DMSO, Aurone 4a, or 4r in 1.5 mL fish system water in 12-well plates. Zebrafish were treated with compound for 2 days, removed from drug for 1 day, and treated for two more days in fresh compound. Animals were imaged at the start and end of treatment using a fluorescence-equipped dissecting microscope at 350 ms exposure. The GFP image was overlaid onto the bright-field image of each animal in Photoshop, and the percent change in leukemia burden was calculated by normalizing the GFP+ area to the total area of the animal in ImageJ.

In Vivo Evaluation of Anti-Cancer Activity and Gross Toxicity in Xenografts.

PC-3 cells suspended in PBS were subcutaneously injected to the lower flanks of immune-deficient nude mice at a density of 2×10⁶ cells/200 ul (PBS). After tumor establishment, 4A formulated in tween80 (5%), DMSO (10%), PEG400 (25%) and PBS (60%) were administered in an intraperitoneal fashion to mice at a daily dose of 10 mg (compound)/kg (mouse). Blank vehicle was used as a control. Tumors and mouse weights were measured and tumor volumes were calculated as Length×width²/2.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

[1] Elhadi, et al., Synthesis and structural elucidation of two new series of aurone derivatives as potent inhibitors against the proliferation of human cancer cells. Med Chem Res 24, 3504-3515 (2015);
[2] Cheng, et al., Design, synthesis and discovery of 5-hydroxyaurone derivatives as growth inhibitors against HUVEC and some cancer cell lines. Eur J Med Chem 45, 5950-5957 (2010).
[3] Zwergel et al., Med Chem Commun 4, 1571-1579 (2013).
[4] Guo et al., Ad Mat Res 781-784, 1235-1239 (2013).
[5] Huang et al., Bioorg Med Chem 15, 5191-5197, doi:10.1016/j.bmc.2007.05.022 (2007).
[6] Pathak, N. P., Ind J App Res 6, 800-802 (2016).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

1. A semisynthetic aurone compound comprising a structure according to Formula (I): 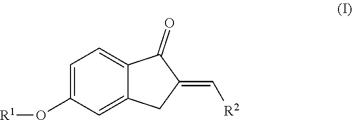
or pharmaceutically acceptable salt thereof,
wherein R¹ is selected from the group consisting of H, an alkyl cyano, an alkyl phenyl substituted with one or more halogens, and combinations thereof; and
wherein R² is selected from the group consisting of an aromatic group, a heterocycle, and a combination thereof.

2. The compound of claim 1, wherein the structure is selected from the group consisting of:

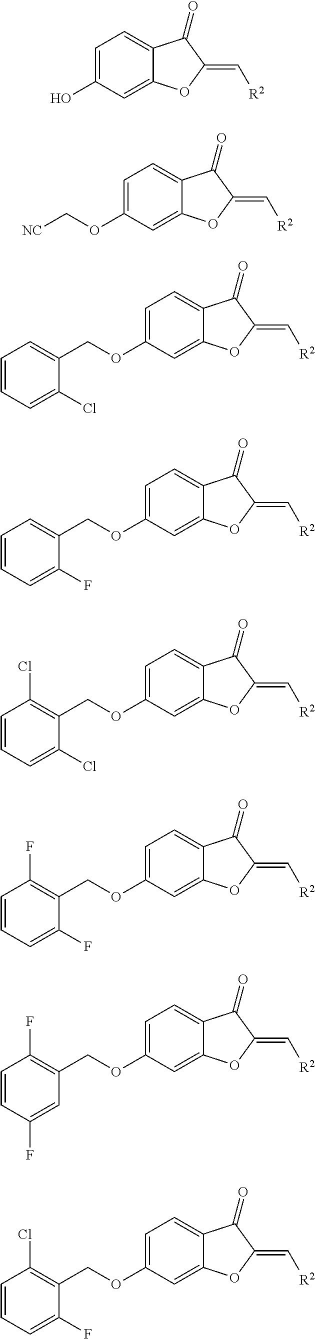

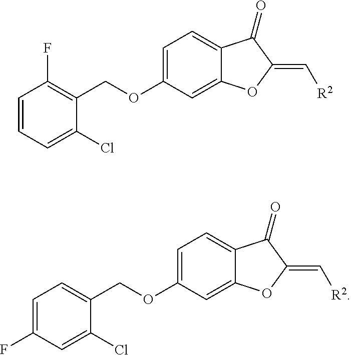

3. The compound of claim 1, wherein R² comprises an aromatic group.

4. The compound of claim 3, wherein the aromatic group comprises at least one substitution selected from the group consisting of a halogen, an alkyl, an alkoxy group, an amino group, a heterocycle, and combinations thereof.

5. The compound of claim 4, wherein the compound comprises the structure according to Formula (II): 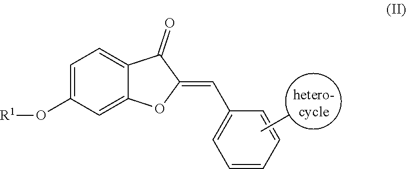
or pharmaceutically acceptable salt thereof.

6. The compound of claim 5, wherein the heterocycle comprises a nitrogen-containing ring.

7. The compound of claim 6, wherein the nitrogen-containing ring comprises a first nitrogen atom and at least one other non-carbon atom selected from the group consisting of nitrogen, oxygen, sulfur, and combinations thereof.

8. The compound of claim 5, wherein the heterocycle comprises 4 to 10 members.

9. The compound of claim 5, wherein the heterocycle is selected from the group consisting of pyridyl, pyridazinyl, pyrrolidyl, morpholinyl, thiomorpholinyl, isoquinolyl, quinolyl, indolyl, and combinations thereof.

10. The compound of claim 5, wherein the heterocycle includes at least one substitution selected from the group consisting of O, OH, a halogen, an alkyl, an alkoxy group, an amino group, a carboxyl group, and combinations thereof.

11. The compound of claim 1, wherein R² comprises a heterocycle.

12. The compound of claim 11, wherein the compound comprises the structure according to Formula (III): 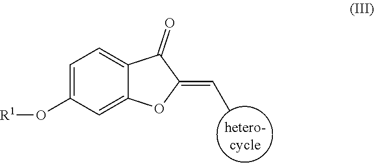
or pharmaceutically acceptable salt thereof.

13. The compound of claim 11, wherein the heterocycle comprises a nitrogen-containing ring.

14. The compound of claim 13, wherein the nitrogen-containing ring comprises a first nitrogen atom and at least one other non-carbon atom selected from the group consisting of nitrogen, oxygen, sulfur, and combinations thereof.

15. The compound of claim 11, wherein the heterocycle comprises 4 to 10 members.

16. The compound of claim 11, wherein the heterocycle is selected from the group consisting of pyridyl, pyridazinyl, pyrrolidyl, morpholinyl, thiomorpholinyl, isoquinolyl, quinolyl, indolyl, and combinations thereof.

17. The compound of claim 11, wherein the heterocycle includes at least one substitution selected from the group consisting of O, OH, a halogen, an alkyl, an alkoxy group, an amino group, a carboxyl group, and combinations thereof.

18. A pharmaceutical composition comprising a compound according to claim 1 and a carrier.

19. A method of treating cancer, the method comprising administering to a patient in need of such treatment an effective amount of a semisynthetic aurone compound according to claim 1.

20. The method of claim 19, wherein the cancer is selected from the group consisting of prostate cancer, colorectal cancer, leukemia, and combinations thereof.
